(19) United States
(12) Patent Application Publication
(10) Pub. No.: US 2025/0377365 A1
(43) Pub. Date: Dec. 11, 2025

(54) METHODS INVOLVING DETECTING TNF STIMULATED GENE 6 (TSG-6) FOR IMPROVING ANTI-TUMOR RESPONSES TO IMMUNE THERAPY IN CANCER PATIENTS

(71) Applicant: BOARD OF REGENTS, THE UNIVERSITY OF TEXAS SYSTEM, Austin, TX (US)

(72) Inventors: Swetha ANANDHAN, Houston, TX (US); Padmanee SHARMA, Houston, TX (US); Sangeeta GOSWAMI, Houston, TX (US)

(73) Assignee: BOARD OF REGENTS, THE UNIVERSITY OF TEXAS SYSTEM, Austin, TX (US)

(21) Appl. No.: 19/127,871

(22) PCT Filed: Nov. 7, 2023
(86) PCT No.: PCT/US2023/078987

§ 371 Date: May 7, 2025

Related U.S. Application Data
(60) Provisional application No. 63/423,401, filed on Nov. 7, 2022.

(51) Int. Cl. G01N 33/68; A61K 31/56; A61K 31/7088; A61K 35/12; A61K 45/06; A61P 35/00; C07K 16/28; G01N 33/50

(52) U.S. Cl. CPC G01N 33/6893; A61K 31/56; A61K 31/7088; A61K 35/12; A61K 45/06; A61P 35/00; C07K 16/2875; G01N 33/5011; G01N 33/5044; G01N 2474/20; G01N 2500/10

(57) ABSTRACT

Aspects herein relate to methods, compositions, and systems directed to the discovery that TSG-6 secretion by cancer-associated fibroblasts (CAFs) in a tumor, in some instances, leads to a poor response to immune checkpoint therapies in the tumor. Inhibition of TSG-6 in certain cancers, in combination with immune checkpoint therapies, led to an improved therapeutic response to the immune checkpoint therapies. Such findings provide methods and compositions for diagnosing, prognosing, and treating certain cancers.

This application claims priority of U.S. Provisional Application No. 63/423,401, filed Nov. 7, 2022, which is hereby incorporated by reference in its entirety.

### BACKGROUND OF THE INVENTION

### I. Field of the Invention

This invention relates to the field of oncology, pathology, and medicine.

### II. Background

Immune checkpoint therapy (ICT) for cancer treatment has drastically transformed clinical outcome of cancer, with patients achieving long-term durable response in cancers such as melanoma. However, tumor types such as pancreatic ductal carcinoma (PDAC) have shown poor response to this treatment. Understanding underlying mechanisms of resistance is critical to develop better and more rational therapeutic combinations with ICT that can promote these “cold tumors” to more “hot tumors”.

ICT resistance in cold tumors has been primarily associated to the presence of dense stromal compartment, abundant immunesuppressive myeloid cells and poor infiltration of T cells in the tumor microenvironment (TME) thereby acting as strong barriers for effective therapy. Tumor and stromal secreted factors are known to play vital roles in inducing such suppressive microenvironments, including the repolarization of myeloid cells.

### SUMMARY OF THE INVENTION

In general, the current invention relates to the discovery that TSG-6 secretion by cancer-associated fibroblasts (CAFs) in a tumor, in some instances, leads to a poor response to immune checkpoint therapies in the tumor. Inhibition of TSG-6 in certain cancers, in combination with immune checkpoint therapies, led to an improved therapeutic response to the immune checkpoint therapies. Such findings provide methods and compositions for diagnosing, prognosing, and treating certain cancers.

Certain aspects related to methods of detecting TNF stimulated gene 6 (TSG-6) secretion in a population of cells. Certain aspects relate to methods of assaying TSG-6 secretion in a population of cells. Certain aspects relate to methods of identifying TSG-6 secretion in a population of cells. In certain aspects, the population of cells is a population of cancer-associated fibroblasts (CAFs). In some aspects, the population of cells are from an individual that has, or is suspected of having cancer. In some aspects, the method comprises the step of detecting TSG-6 in in a biological sample. The biological sample may comprise CAF secretions, including CAF secretions from an individual. The biological sample may comprise CAFs from an individual. The individual may have, or is suspected of having, a cancer. The cancer may be a candidate for an immune checkpoint therapy. Cancers that are candidates for an immune checkpoint therapy are known in the art, such as any cancer described herein. In some aspects, the biological sample, including those comprising CAF secretions and/or comprising CAFs, comprises a tumor biopsy. The tumor biopsy may be a solid tumor biopsy. The tumor biopsy may be collected in any manner described herein.

In some aspects, the biological sample is enriched for fibroblasts. In certain aspects, the biological sample is enriched for CAFs. The enrichment may be by centrifugation, affinity purification, cell sorting, or any other method known in the art. In certain aspects, the biological sample comprises a supernatant from a sample, such sample may comprise CAFs.

In certain aspects, the biological sample is prepared for immunohistochemistry. The biological sample may be fixed in a particular medium suitable for immunohistochemistry. The biological sample may be formalin fixed and sectioned. In certain aspects, detecting TSG-6 comprises detecting by immunohistochemistry. In some aspects, the detecting by immunohistochemistry comprises contacting the biological sample, which may be fixed and/or stained, with an antibody and/or reagent capable of staining the TSG-6 present in the biological sample.

In certain aspects, detecting TSG-6 comprises detecting TSG-6 protein. Detecting TSG-6 protein comprises detecting by immunohistochemistry. In some aspects, detecting TSG-6 protein comprises detecting by Western blot, ELISA, cytometry, chromatography, mass spectrometry, and/or immunoprecipitation.

In certain aspects, detecting TSG-6 comprises detecting TSG-6 mRNA. Detecting TSG-6 mRNA may comprise any method of detecting specific mRNA, including by polymerase chain reaction (PCR). The PCR may be a reverse transcriptase PCR (RT-PCR). The PCR may be quantitative PCR (qPCR). The PCR may be quantitative RT-PCR (RT-qPCR). In certain aspects, detecting TSG-6 mRNA comprises sequencing. The sequencing may be any sequencing suitable for determining the expression profile of a cell. In certain aspects, the sequencing comprises RNA sequencing. The RNA sequencing may comprise single cell RNA sequencing. In certain aspects, the immune checkpoint therapy comprises an anti-PD1 composition and/or an anti-CTLA4 composition, or other agent capable of inhibiting or suppressing the immune checkpoint machinery.

In certain aspects, the detecting TSG-6 comprises detecting an amount of TSG-6 that is greater than a reference level. In certain aspects, the detecting TSG-6 comprises detecting an amount of TSG-6 that is substantially equal to or less than a reference level. In some aspects, the reference level is determined by measuring TSG-6 in two or more biological samples from the individual. The reference level may comprise an amount of TSG-6 found in an individual responsive to an immune checkpoint therapy or a healthy individual. The reference level may comprise an amount of TSG-6 found in a cohort of individuals, including a cohort of individuals responsive to an immune checkpoint therapy or a cohort of healthy individuals.

In certain aspects, detecting TSG-6 comprises detecting any amount of a TSG-6 gene product, including any detectable amount of a TSG-6 gene product. In certain aspects, detecting TSG-6 comprises detecting an amount of TSG-6 over a background level. In certain aspects, detecting TSG-6 comprises detecting an amount of TSG-6 over a standard level. In certain aspects, the standard level comprises a level of TSG-6 in a non-cancerous cell. In certain aspects, the standard level is an average level of TSG-6 present in a population of cancer cells known to be sensitive to a cancer therapy (including any cancer therapy disclosed herein such as an immune checkpoint inhibitor). In certain aspects, the standard level is an average level of TSG-6 present in a population of cancer cells known to be refractory to a cancer therapy (including any cancer therapy disclosed herein such as an immune checkpoint inhibitor). In certain aspects, one skilled in the art can determine the standard level using known pre-clinical or clinical techniques. In certain aspects, one skilled in the art can determine a standard level by measuring TSG-6 in a variety of individuals (such as patients in a clinical trial).

Certain aspects relate to methods of treating a cancer in an individual. Certain aspects relate to method of reducing the severity of a cancer in an individual. Certain aspects relate to methods of preventing symptoms associated with cancer in an individual. In some aspects, the method comprises the step of administering a non-immune checkpoint therapy to an individual who has been determined to have cancer-associated fibroblasts (CAFs) that secrete TSG-6 in an amount greater than a reference level. The non-immune checkpoint therapy may comprise a chemotherapy, a non-immune checkpoint immunotherapy, a cell therapy, a biologic, a steroid, radiation, and/or surgery. In certain aspects, the non-immune checkpoint therapy comprises a cancer therapy. In certain aspects, the reference level is any amount, including a detectable amount, of TSG-6 secreting CAFs, which may be present in the individual. In certain aspects, the reference level is determined by measuring TSG-6 secreting CAFs in two or more biological samples from the individual. The biological samples may have been taken before, during, or after a therapy. The biological sample may have been taken prior to the individual being diagnosed with cancer. In certain aspects, the reference level comprises an amount of TSG-6 secreting CAFs found in an individual responsive to an immune checkpoint therapy or a healthy individual. The individual may be considered responsive to the immune checkpoint therapy, or any other therapy described herein, when the therapy causes the individual to have a reduction in symptoms, reduction in tumor burden, reduction in tumor aggressiveness, reduction in tumor size, reduction in metastasis, and/or improvement in quality of life. A healthy individual may be an individual that does not have cancer or does not have cancer that is refractory to an immune checkpoint therapy.

In some aspects, the method comprising the step of administering an immune checkpoint therapy to an individual who has been determined to have cancer-associated fibroblasts (CAFs) that secrete TSG-6 in an amount substantially equal to or less than a reference level. The biological samples may have been taken before, during, or after a therapy. The biological sample may have been taken prior to the individual being diagnosed with cancer. In certain aspects, the reference level is a detectable amount of TSG-6 secreting CAFs present in the individual. In certain aspects, the reference level is determined by measuring TSG-6 secreting CAFs in two or more biological samples from the individual. In certain aspects, the reference level comprises an amount of TSG-6 secreting CAFs found in an individual responsive to an immune checkpoint therapy or a healthy individual. The individual may be considered responsive to the immune checkpoint therapy, or any other therapy described herein, when the therapy causes the individual to have a reduction in symptoms, reduction in tumor burden, reduction in tumor aggressiveness, reduction in tumor size, reduction in metastasis, and/or improvement in quality of life. A healthy individual may be an individual that does not have cancer or does not have cancer that is refractory to an immune checkpoint therapy.

Certain aspects relate to methods of prognosing the effectiveness of an immune checkpoint therapy in an individual. Certain aspects relate to methods for evaluating effectiveness of an immune checkpoint therapy in a cancer patient, the method comprising the step of detecting TSG-6 in a biological sample to get a measurement and comparing the measurement to a reference. In some aspects, the individual is administered a therapy, including any therapy disclosed herein, such as a therapy that targets TSG-6, an immune checkpoint therapy, or a non-immune checkpoint therapy, based on the measurement and/or comparison of the measurement to the reference. In certain aspects, the immune checkpoint therapy comprises an anti-PD1 composition and/or an anti-CTLA4 composition.

In certain aspects, the biological sample comprises CAF secretions. In certain aspects, the biological sample comprises CAFs. In certain aspects, the biological sample comprises a tumor biopsy. In some aspects, the biological sample is enriched for fibroblasts, which may be CAFs. The biological sample may be prepared for the detecting, including by prepared for immunohistochemistry. In certain aspects, detecting TSG-6 comprises detecting TSG-6 protein. Detecting TSG-6 protein comprises detecting by immunohistochemistry. In some aspects, detecting TSG-6 protein comprises detecting by Western blot, ELISA, cytometry, chromatography, mass spectrometry, and/or immunoprecipitation.

In certain aspects, detecting TSG-6 comprises detecting TSG-6 mRNA. Detecting TSG-6 mRNA may comprise any method of detecting specific mRNA, including by polymerase chain reaction (PCR). The PCR may be a reverse transcriptase PCR (RT-PCR). The PCR may be quantitative PCR (qPCR). The PCR may be quantitative RT-PCR (RT-qPCR). In certain aspects, detecting TSG-6 mRNA comprises sequencing. The sequencing may be any sequencing suitable for determining the expression profile of a cell. In certain aspects, the sequencing comprises RNA sequencing. The RNA sequencing may comprise single cell RNA sequencing.

In certain aspects, the reference is a detectable amount of TSG-6 secreting CAFs present in the individual. In certain aspects, the reference is determined from measuring TSG-6 secreting CAFs in two or more biological samples from the individual. In certain aspects, the reference comprises an amount of TSG-6 secreting CAFs found in an individual responsive to an immune checkpoint therapy or a healthy individual. The individual may be considered responsive to the immune checkpoint therapy, or any other therapy described herein, when the therapy causes the individual to have a reduction in symptoms, reduction in tumor burden, reduction in tumor aggressiveness, reduction in tumor size, reduction in metastasis, and/or improvement in quality of life. A healthy individual may be an individual that does not have cancer or does not have cancer that is refractory to an immune checkpoint therapy.

Certain aspects relate to methods of treating a cancer in an individual. In some aspects, the method comprises the step of administering a composition comprising a therapeutically effective amount of an anti-TSG-6 agent. In certain aspects, the individual has been determined to have cancer-associated fibroblasts (CAFs) that secrete TSG-6 in an amount greater than a reference level. In some aspects, the reference level is a detectable amount of TSG-6 secreting CAFs present in the individual. In some aspects, the reference level is determined by measuring TSG-6 secreting CAFs in two or more biological samples from the individual. In certain aspects, the reference level comprises an amount of TSG-6 secreting CAFs found in an individual responsive to an immune checkpoint therapy or a healthy individual. In certain aspects, the anti-TSG-6 composition comprises an antibody targeting TSG-6, an RNA interference composition targeting TSG-6, and/or a small molecule capable of inhibiting TSG-6. In certain aspects, the method further comprises administering a therapeutically effective amount of an immune checkpoint therapy. In certain aspects, the composition comprises the immune checkpoint therapy. In certain aspects, the composition does not comprise the immune checkpoint therapy. In certain aspects, the composition and immune checkpoint therapy are given concurrently. The compositions given concurrently may be in the same composition or in separate compositions. Concurrently may be that the anti-TSG-6 agent is given within 1, 2, 3, 4, 5, 6, 7, 8, 9, 10 or more, or any range derivable therein, minutes, hours, or days of the immune checkpoint therapy. In certain aspects, the composition and immune checkpoint therapy are given sequentially. In certain aspects, the immune checkpoint therapy comprises an anti-PD1 composition and/or an anti-CTLA4 composition. In certain aspects, the cancer is refractory to and immune checkpoint therapy. In certain aspects, the cancer is a myeloid-rich cancer. In certain aspects, the cancer is a pancreatic cancer (which may be a pancreatic ductal adenocarcinoma), a glioblastoma, and/or a prostate cancer. In certain aspects, the cancer is a melanoma. In some aspects, administration of the anti-TSG-6 composition, alone or in combination with one or more immune checkpoint therapies, to an individual with a tumor alters cells infiltrating into the tumor. The cells may be immune cells, such as monocytes, MHCII⁺ macrophages, MHCII⁻ macrophages, dendritic cells, neutrophils, T-cells, NK cells, and/or B-cells. The T-cells may be T_{reg} cells, CD4⁺ cells, and/or CD8⁺ cells.

Certain aspects relate to methods for determining a treatment for an individual with cancer, the method comprising the step of administering a non-checkpoint inhibitor therapy to an individual found to have an amount of TSG-6 secreting cancer-associated fibroblasts (CAFs) equal to or greater than a reference. In certain aspects, the detectable amount of TSG-6 secreting CAFs is found by measuring the expression of TSG-6 in at least one biological sample from the individual. In certain aspects, the biological sample comprises a tumor biopsy. In certain aspects, the biological sample is enriched for fibroblasts. In certain aspects, the biological sample is enriched CAFs. In certain aspects, the biological sample is prepared for immunohistochemistry. In certain aspects, the reference is a detectable amount of TSG-6 secreting CAFs present in the individual. In certain aspects, the reference is determined from measuring TSG-6 secreting CAFs in two or more biological samples from the individual. In certain aspects, the reference comprises an amount of TSG-6 secreting CAFs found in an individual responsive to an immune checkpoint therapy or a healthy individual.

Methods included herein can comprise 1, 2, 3, 4, 5, 6 or more steps, including any of the following: detecting TSG-6 in a biological sample comprising CAF secretions, comparing a measured level of TSG-6 to a reference, administering a non-immune checkpoint therapy, administering an immune checkpoint therapy, observing an individual that has received a therapy, and changing a treatment plan for the individual.

The following definitions may be used in describing the invention and claims.

As used herein, an “individual” can refer to a human. In certain aspects, the individual is a human patient, including a human cancer patient. In certain aspects, the individual has, is diagnosed with having, is determined to have, or is suspected of having cancer.

As used herein, tumors that are “sensitive” to an immune checkpoint therapy (ICT) are tumors have a detectable decrease in tumor size after completion of an ICT regimen. Conversely, tumors that are “resistant” to an ICT are tumors that have no detectable decrease in tumor size after completion of an ICT regimen.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.” Any term used in singular form also comprise plural form and vice versa.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an aspect or aspect.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments and aspects described in the context of the term “comprising” may also be implemented in the context of the term “consisting of” or “consisting essentially of.”

It is contemplated that any aspect discussed in this specification can be implemented with respect to any method or composition of the invention, and vice versa. Furthermore, compositions of the invention can be used to achieve methods of the invention.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other aspects and embodiments are discussed throughout this application. Any embodiment or aspect discussed with respect to one aspect of the disclosure applies to other aspects of the disclosure as well and vice versa.

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also aspects that may be implemented in the context of aspects discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific aspects presented herein.

FIGS. 1A-1G show TSG6/Tnfaip6 is expressed by cancer-associated fibroblasts in ICT resistant pancreatic tumors. (A) Schematic representation of scRNAseq experimental design. (B) UMAP plot of sorted intratumoral CD45 negative cells. (C) UMAP plots indicating marker gene expression for B16F10 tumor cells (pmel, mlana), MT4 tumor cells (krt18, krt19) and fibroblasts (col1a1, dcn). (D) UMAP representation highlighting differences in fibroblast abundance between mT4 and B16F10 tumors (green circle). (E) MCP scoring of fibroblast abundance in The Cancer Genome Atlas (TCGA) data set between melanoma (skcm, skin cutaneous melanoma) and pancreatic tumors (paad, pancreatic adenocarcinoma). (F) UMAP plot highlighting tnfaip6 (protein: TSG-6) expression in murine B16F10 and mT4 tumors. (G) Analysis of TNFAIP6 RNA expression in TCGA data set between melanoma (skcm, skin cutaneous melanoma) and pancreatic tumors (paad, pancreatic adenocarcinoma).

FIGS. 2A-2H show ICT resistant pancreatic tumors are dominated by suppressive myeloid cells whereas ICT sensitive melanoma tumors are dominant by immune stimulatory myeloid cells. (A) Schematic representation of scRNAseq experimental design. (B) Representation UMAP plot of intratumoral immune cell landscape in B16F10 and MT4 tumors. 3 tumors in each were pooled for internal control. All major immune cell subsets were identified. (C) Characterization of immune subsets identified in FIG. 1B based on their marker gene expressions. (D) Cluster frequency ratios plot of each immune subset in B16F10 and MT4 tumors. (E) Immune cell populations identified via CyTOF analysis performed on murine mT4 pancreatic tumors (n=6) and B16F10 melanoma tumors (n=5). Based on the markers, the populations were defined as CD8 T cells (CD8+CD3e+), CD4 T cells (CD4+CD3e+), Tregs cells (FoxP3+ CD4+CD3e+), B cells (CD19+ cells), NK cells (NK1.1+), macrophages (CD11b+ F4/80+), monocytes (CD11b+Ly6c+F4/80−), neutrophils (CD11b+Ly6C+Ly6G+), dendritic cells (DCs) (CD11c+MHCII+). (F) UMAP representation of total macrophage, monocyte, and neutrophil subsets in B16F10 tumors and mT4 to indicate minimal overlap between subsets. (G) Heatmap of functional markers for the individual myeloid subsets providing phenotypic information. Expression levels are scaled between minimum and maximum expression for each gene across all clusters. (H) GSEA analysis between mT4 and B16F10 macrophages. Circles indicate the total no. of genes associated with each specific pathway. Expression levels are scaled between minimum and maximum expression for each gene across all clusters.

FIGS. 3A-3C show CAF secreted TSG-6 interacts with suppressive myeloid cells specifically in the ICT resistant pancreatic tumors. (A) Heatmap indicting expression of tnfaip6 (TSG-6) and receptor Cd44 across the indicated cell subsets across B16F10 (red bar) and mT4 tumors (blue bar), (B) Representative plot of tnfaip6 interaction with Cd44 expressing myeloid cells in B16F10 and mT4 tumors using Cell chat analysis. Circles indicate p values and color indicate levels of expression. Expression levels are scaled between minimum and maximum expression for each gene across all clusters. (C) Fold change of indicated gene expression on the bone marrow derived macrophages stimulated with either LPS or recombinant TSG-6via qPCR. Statistical significance was calculated using Student's t-test (two-tailed). P values for each comparison has been indicated in the figure. ns, not significant.

FIGS. 4A-4E show blocking TSG-6 reinvigorates the pancreatic tumor microenvironment and improves ICT efficacy in mice (A) Representative experimental plan for in vivo antibody blocking studies. (B) Survival plot indicating therapeutic activity of anti-TSG-6, anti-CTLA-4 and anti-PD-1. Data cumulative of three independent experiments. Statistical significance was calculated using Mantel-Cox test. (C) UMAP representation indicating immune cells identified upon CyTOF staining. (D) Heatmap indicating expression of proteins analyzed in the CyTOF experiment. (E) Abundance of indicated cell clusters as a proportion of total CD45+ cells.

FIGS. 5A-5D show (A) UMAP plot of tumor and stromal compartment of human pancreatic tumors scRNAseq data from 24 PDAC patients and 11 normal pancreas (right) with cells subsets defined based on their marker gene expressions (left). (B) Expression of TNFAIP6 across normal pancreas and tumors. (C) Representative multi-immunofluorescence (mIF) images highlighting presence of TSG-6 protein in human pancreatic and melanoma FFPE sample. White arrows highlight the TSG6+SMA+ cells in the pancreatic TME which are not seen in the melanoma tumors. (D) Quantification of SMA+ cells (right) and TSG6 expressing SMA+ cells (left) mIF images after TSG-6 staining in the above samples (n=8 to 9 in each tumor type). (E) Representative multi-immunofluorescence (mIF) image highlighting co-localization of CD68+CD44+CD163+ myeloid cells with TSG-6 (white arrow) in human pancreatic tissue FFPE samples (n=4). (F) Analysis of the mIF images using infiltration analysis technique. Red borders indicate TSG6+ cells and red to green indicate the distance from the borders to outside. Number of CD68+CD44+ cells that were at a distance of 0-20 um from TSG6+ cells were quantified and plotted (right).

FIGS. 6A-6C show (A) Plot of total no. of cells analyzed from each group for scRNAseq analysis. (B) Representative UMAP plot indicating abundance of each cell cluster across B16F10 and mT4 tumors. (C) UMAP plots (right) and quantification (left) total T cells (light gray) and myeloid cells (dark gray) present in both tumor models.

FIGS. 7A-7B show (A) Table indicting patient characteristics used for mIF. (B) Analysis of TNFAIP6 expression in The Cancer Genome Atlas (TCGA) data set in some ICT more-responsive tumor types (green shades) and less-responsive tumor types (red shades)/ acc, adrenocortical carcinoma; blca, bladder urothelial carcinoma; cesc, cervical squamous cell carcinoma and endocervical adenocarcinoma; lihc, liver hepatocellular carcinoma; skcm, skin cutaneous melanoma; uvm, uveal melanoma; gbm, glioblastoma; paad, pancreatic adenocarcinoma; sarc, sarcoma.

### DETAILED DESCRIPTION OF THE INVENTION

### I. General Aspects

Certain aspects herein relate to the identification of a novel mediator, TNF stimulated Gene 6 (TSG-6), which plays a role in regulating the phenotype of myeloid cells in the tumor microenvironment and promotes resistance to immune checkpoint therapy (ICT) in myeloid-rich tumors. Some aspects of the disclosure show TSG-6 is expressed by cancer-associated fibroblasts and interacts with CD44 expressing suppressive myeloid cells in the tumor. Certain aspects show validation these observations in human pancreatic tumors. In certain aspects, murine studies were performed in ICT-resistant pancreatic tumors and show that targeting TSG-6 can improve ICT efficacies in pancreatic tumors. Thus, in some aspects, targeting of TSG-6 in combination with ICT could have significant clinical impact in ICT non-responsive tumors such as pancreatic cancer.

Certain aspect herein identified a novel mediator TNF-Stimulated Factor 6 (TSG-6) which is expressed by cancer-associated fibroblasts (CAFs) in the tumors. High-throughput techniques, such as single cell RNA sequencing (scRNAseq) and mass cytometry (CyTOF), were performed in some in vivo mechanistic aspects using ICT sensitive melanoma model (B16F10) and resistant pancreatic model (MT4). Such aspects provide evidence that TSG-6 interacts with the CD44+ suppressive myeloid cells in the TME. These findings were also confirmed in patient samples, in some aspects. Importantly, in vivo neutralization of TSG-6 improves ICT response and provides survival benefit in mice, in certain aspects. Overall, certain aspects herein identified novel mechanisms of action in ICT-resistant tumors that offer significant potential for combinatorial targeting with immune checkpoint therapy and provide clinical benefit.

### II. Therapeutic Methods

Methods of the disclosure relate to treating subjects and patients with a cancer therapy. The cancer therapy may be one described herein, including an immune checkpoint therapy and/or an agent that targets TSG-6, and may be given with respect to a patient having been determined to have a certain biomarker profile. The agent that targets TSG-6 may be an anti-TSG-6 antibody, an RNA interference composition that targets TSG-6, or an inhibitor of TSG-6. The RNA interference composition may be an siRNA, miRNA, or other noncoding RNA. The biomarker profile may be the presence or absence of one or more biomarkers expressed on or by a cancer-associated fibroblast (CAF). The biomarker may comprise all or part of TSG-6. In some aspects, the therapy described below is given to a patient with a poor prognosis, unfavorable prognosis, or to a patient determined to be high risk. In some aspects, the therapy is given to a patient having a prognosis that reflects a favorable outcome for an immune checkpoint therapy. In some aspects, the therapy described below is given to a patient with a favorable prognosis, or to a patient determined to be low risk. In some aspects, the therapy is given to a patient having a prognosis that reflects an unfavorable outcome for an immune checkpoint therapy. Also contemplated are combinations of the therapies described below.

The compositions of the disclosure, including agents that target TSG-6, may be used for in vivo, in vitro, or ex vivo administration. The route of administration of the composition may be, for example, intratumoral, intracutaneous, subcutaneous, intravenous, local, topical, and intraperitoneal administrations.

### A. Immune Checkpoint Therapies and Combination Treatment

Aspects of the disclosure may include administration of immune checkpoint therapies, also known as checkpoint inhibitors, which are further described below. The administration of the immune checkpoint therapies may be done in combination with one or more other therapies, such as an agent that targets TSG-6. In some aspects, pharmacological drugs, biologics, nucleic acids, and/or blocking antibodies are generated against TSG-6 which in combination with an immune checkpoint therapy will improve clinical response in patients, including those with myeloid rich tumors like pancreatic tumors, glioblastoma and prostate cancer. In some aspects, TSG-6 is used to develop immunohistochemistry assays that will enable selection patients who should receive, and/or may be responsive to, anti-TSG-6 optionally with an immune checkpoint therapy.

### 1. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some aspects, PD-1, PDL1, and PDL2 are human PD-1, PDL1 and PDL2.

In some aspects, the PD-1 inhibitor is a molecule that inhibits the binding of PD-1 to its ligand binding partners. In a specific aspect, the PD-1 ligand binding partners are PDL1 and/or PDL2. In another aspect, a PDL1 inhibitor is a molecule that inhibits the binding of PDL1 to its binding partners. In a specific aspect, PDL1 binding partners are PD-1 and/or B7-1. In another aspect, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some aspects, the PD-1 inhibitor is an anti-PD-1 antibody (e.g., a human antibody, a humanized antibody, or a chimeric antibody). In some aspects, the anti-PD-1 antibody is selected from the group consisting of nivolumab, pembrolizumab, and pidilizumab. In some aspects, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some aspects, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some aspects, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some aspects, the inhibitor comprises the heavy and light chain CDRs or VRs of nivolumab, pembrolizumab, or pidilizumab. Accordingly, in one aspect, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another aspect, the antibody competes for binding with and/or binds to the same epitope on PD-1, PDL1, or PDL2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

### 2. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some aspects, the inhibitor blocks the CTLA-4 and B7-1 interaction. In some aspects, the inhibitor blocks the CTLA-4 and B7-2 interaction.

In some aspects, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (sec, e.g., WO0 1/14424).

In some aspects, the inhibitor comprises the heavy and light chain CDRs or VRs of tremelimumab or ipilimumab. Accordingly, in one aspect, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another aspect, the antibody competes for binding with and/or binds to the same epitope on PD-1, B7-1, or B7-2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

### B. Administration of Therapeutic Compositions

The therapy provided herein may comprise administration of a combination of therapeutic agents, including a first therapy, such as an immune checkpoint therapy, and a second therapy, such as an agent that targets TSG-6. The therapies may be administered in any suitable manner known in the art. For example, the first and second cancer treatment may be administered sequentially (at different times) or concurrently (at the same time). In some aspects, the first and second cancer treatments are administered in a separate composition. In some aspects, the first and second cancer treatments are in the same composition.

In some aspects, the first therapy and the second therapy are administered substantially simultaneously. In some aspects, the first therapy and the second therapy are administered sequentially. In some aspects, the first therapy, the second therapy, and a third therapy are administered sequentially. In some aspects, the first therapy is administered before administering the second therapy. In some aspects, the first therapy is administered after administering the second therapy.

Aspects of the disclosure relate to compositions and methods comprising therapeutic compositions. In some aspects, the therapeutic composition comprises an immune checkpoint therapy. In some aspects, the therapeutic composition comprises an anti-TSG-6 agent. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some aspects, the cancer therapy is administered intravenously, intramuscularly, subcutaneously, topically, orally, transdermally, intraperitoneally. intraorbitally, by implantation, by inhalation, intrathecally, intraventricularly, or intranasally. In some aspects, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

In some aspects, the first therapy is administered at a dose of between 1 mg/kg and 5000 mg/kg, or any range derivable therein. In some aspects, the first therapy is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 mg/kg, or any range derivable therein.

In some aspects, a single dose of the second therapy is administered. In some aspects, multiple doses of the second therapy are administered. In some aspects, the second therapy is administered at a dose of between 1 mg/kg and 5000 mg/kg, or any range derivable therein. In some aspects, the second therapy is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 mg/kg, or any range derivable therein.

The quantity to be administered, both according to number of treatments and unit dose, depends on the treatment effect desired. An effective dose is understood to refer to an amount necessary to achieve a particular effect. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain aspects, the effective dose of the pharmaceutical composition is one which can provide a blood level of about 1 μM to 150 μM. In another aspect, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 M; or about 25 μM to 150 μM; or about 25 μM to 100 μM; or about 25 μM to 50 μM; or about 50 μM to 150 μM; or about 50 μM to 100 μM (or any range derivable therein). In other aspects, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain aspects, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### C. Cancer Therapy

In some aspects, the method further comprises administering a cancer therapy to the patient. The cancer therapy may be chosen based on the expression level measurements of TSG-6, alone or in combination with a separate clinical risk score calculated for the patient. In some aspects, the cancer therapy comprises a local cancer therapy. In some aspects, the cancer therapy excludes a systemic cancer therapy. In some aspects, the cancer therapy excludes a local therapy. In some aspects, the cancer therapy comprises a local cancer therapy without the administration of a system cancer therapy. In some aspects, the cancer therapy comprises an immunotherapy, which may be an immune checkpoint therapy. Any of these cancer therapies may also be excluded. Combinations of these therapies may also be administered.

The term “cancer,” as used herein, may be used to describe a solid tumor, metastatic cancer, or non-metastatic cancer. In certain aspects, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus. In some aspects, the cancer is recurrent cancer. In some aspects, the cancer is Stage I cancer. In some aspects, the cancer is Stage II cancer. In some aspects, the cancer is Stage III cancer. In some aspects, the cancer is Stage IV cancer.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; amcloblastic odontosarcoma; ameloblastoma, malignant; ameloblastic fibrosarcoma; pincaloma, malignant; chordoma; glioma, malignant; cpendymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

### D. Pharmaceutical Compositions

In certain aspects, the compositions or agents for use in the methods, such as an immune checkpoint therapy and/or an agent that targets TSG-6, are suitably contained in a pharmaceutically acceptable carrier. The carrier is non-toxic, biocompatible and is selected so as not to detrimentally affect the biological activity of the agent. The agents in some aspects of the disclosure may be formulated into preparations for local delivery (i.e. to a specific location of the body, such as a tumor or other tissue) or systemic delivery, in solid, semi-solid, gel, liquid or gaseous forms such as tablets, capsules, powders, granules, ointments, solutions, depositories, inhalants and injections allowing for oral, parenteral or surgical administration. Certain aspects of the disclosure also contemplate local administration of the compositions by coating medical devices and the like.

Suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose any biocompatible oil may be employed including synthetic mono-or diglycerides. In addition, fatty acids such as oleic acid find use in the preparation of injectables. The carrier and agent may be compounded as a liquid, suspension, polymerizable or non-polymerizable gel, paste or salve.

The carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting examples, microparticles, microspheres, nanospheres or nanoparticles composed of proteins, liposomes, carbohydrates, synthetic organic compounds, inorganic compounds, polymeric or copolymeric hydrogels and polymeric micelles.

In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

Solutions of pharmaceutical compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

In certain aspects, the pharmaceutical compositions are advantageously administered in the form of injectable compositions either as liquid solutions or suspensions; solid forms suitable or solution in, or suspension in, liquid prior to injection may also be prepared. These preparations also may be emulsified. A typical composition for such purpose comprises a pharmaceutically acceptable carrier. For instance, the composition may contain 10 mg or less, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, antgifungal agents, anti-oxidants, chelating agents and inert gases. The pH and exact concentration of the various components the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

In further aspects, the pharmaceutical compositions may include classic pharmaceutical preparations. Administration of pharmaceutical compositions according to certain aspects may be via any common route so long as the target tissue is available via that route. This may include oral, nasal, buccal, rectal, vaginal, tumoral, or topical. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, aerosol delivery can be used. Volume of the aerosol may be between about 0.01 ml and 0.5 ml, for example.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

In some aspects, pharmaceutical compositions are administered to a subject. Different aspects may involve administering an effective amount of a composition to a subject. In some aspects, an antibody or antigen binding fragment capable of binding to [protein of interest] may be administered to the subject to protect against or treat a condition (e.g., cancer). Alternatively, an expression vector encoding one or more such antibodies or polypeptides or peptides may be given to a subject as a preventative treatment. Additionally, such compositions can be administered in combination with an additional therapeutic agent (e.g., a chemotherapeutic, an immunotherapeutic, a biotherapeutic, etc.). Such compositions will generally be dissolved or dispersed in a pharmaceutically acceptable carrier or aqueous medium.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions, such as any antibody described herein, may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### III. Diagnostic and Prognostic Methods

Certain aspects relate to diagnosing or prognosing an individual. The methods may be used to determine the effectiveness of one or more therapies in treating cancer in the individual. The therapies may comprise an immune checkpoint therapy and/or an agent that targets TSG-6.

### A. Sample Preparation

In certain aspects, methods involve obtaining a sample from a subject. The subject may have, or be suspected of having cancer. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. In other aspects the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

In certain aspects, the sample is prepared for

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain aspects the samples are obtained by biopsy. In other aspects the sample is obtained by swabbing, endoscopy, scraping, phlebotomy, or any other methods known in the art. In some cases, the sample may be obtained, stored, or transported using components of a kit of the present methods. In some cases, multiple samples, such as multiple esophageal samples may be obtained for diagnosis by the methods described herein. In other cases, multiple samples, such as one or more samples from one tissue type (for example esophagus) and one or more samples from another specimen (for example serum) may be obtained for diagnosis by the methods. In some cases, multiple samples such as one or more samples from one tissue type (e.g. esophagus) and one or more samples from another specimen (e.g. serum) may be obtained at the same or different times. Samples may be obtained at different times are stored and/or analyzed by different methods. For example, a sample may be obtained and analyzed by routine staining methods or any other cytological analysis methods.

In some aspects the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some aspects, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one aspect, the sample is a fine needle aspirate of a esophageal or a suspected esophageal tumor or neoplasm. In some cases, the fine needle aspirate sampling procedure may be guided by the use of an ultrasound, X-ray, or other imaging device.

In some aspects of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some aspects of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some aspects, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### B. Protein Assays

A variety of techniques can be employed to measure expression levels of polypeptides and proteins, including TSG-6, in a biological sample to determine biomarker expression levels. Examples of such formats include, but are not limited to, enzyme immunoassay (EIA), radioimmunoassay (RIA), Western blot analysis, immunohistochemistry, and enzyme linked immunoabsorbant assay (ELISA). A skilled artisan can readily adapt known protein/antibody detection methods for use in determining protein expression levels of biomarkers.

In one aspect, antibodies, or antibody fragments or derivatives, can be used in methods such as Western blots, ELISA, or immunofluorescence techniques to detect biomarker expression. In some aspects, either the antibodies or proteins are immobilized on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present disclosure. The support can then be washed with suitable buffers followed by treatment with the detectably labeled antibody. The solid phase support can then be washed with the buffer a second time to remove unbound antibody. The amount of bound label on the solid support can then be detected by conventional means.

Immunohistochemistry methods are also suitable for detecting the expression levels of biomarkers. In some aspects, antibodies or antisera, including polyclonal antisera, and monoclonal antibodies specific for each marker may be used to detect expression. The antibodies can be detected by direct labeling of the antibodies themselves, for example, with radioactive labels, fluorescent labels, hapten labels such as, biotin, or an enzyme such as horseradish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

Immunological methods for detecting and measuring complex formation as a measure of protein expression using either specific polyclonal or monoclonal antibodies are known in the art. Examples of such techniques include enzyme-linked immunosorbent assays (ELISAs), radioimmunoassays (RIAs), fluorescence-activated cell sorting (FACS) and antibody arrays. Such immunoassays typically involve the measurement of complex formation between the protein and its specific antibody. These assays and their quantitation against purified, labeled standards are well known in the art. A two-site, monoclonal-based immunoassay utilizing antibodies reactive to two non-interfering epitopes or a competitive binding assay may be employed.

Numerous labels are available and commonly known in the art. Radioisotope labels include, for example, 36S, 14C, 1251, 3H, and 1311. The antibody can be labeled with the radioisotope using the techniques known in the art. Fluorescent labels include, for example, labels such as rare earth chelates (europium chelates) or fluorescein and its derivatives, rhodamine and its derivatives, dansyl, Lissamine, phycoerythrin and Texas Red are available. The fluorescent labels can be conjugated to the antibody variant using the techniques known in the art. Fluorescence can be quantified using a fluorimeter. Various enzyme-substrate labels are available and U.S. Pat. Nos. 4,275,149, 4,318,980 provides a review of some of these. The enzyme generally catalyzes a chemical alteration of the chromogenic substrate which can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2.3-dihydrophthalazinediones, malate dehydrogenase, urease, peroxidase such as horseradish peroxidase (HRPO), alkaline phosphatase, .beta.-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for Use in Enzyme Immunoassay, in Methods in Enzymology (Ed. J. Langone & H. Van Vunakis), Academic press, New York, 73:147-166 (1981).

In some aspects, a detection label is indirectly conjugated with an antibody. The skilled artisan will be aware of various techniques for achieving this. For example, the antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten (e.g., digoxin) and one of the different types of labels mentioned above is conjugated with an anti-hapten antibody (e.g., anti-digoxin antibody). In some aspects, the antibody need not be labeled, and the presence thereof can be detected using a labeled antibody, which binds to the antibody.

### C. Monitoring

In certain aspects, the biomarker-based method may be combined with one or more other cancer diagnosis or screening tests at increased frequency if the patient is determined to be at high risk for recurrence or have a poor prognosis based on the biomarker as described above.

In some aspects, the methods of the disclosure further include one or more monitoring tests. The monitoring protocol may include any methods known in the art. In particular,

the monitoring include obtaining a sample and testing the sample for diagnosis. For example, the monitoring may include endoscopy, biopsy, laparoscopy, colonoscopy, blood test, genetic testing, endoscopic ultrasound, X-ray, barium enema x-ray, chest x-ray, barium swallow, a CT scan, a MRI, a PET scan, or HER2 testing. In some aspects, the monitoring test comprises radiographic imaging. Examples of radiographic imaging this is useful in the methods of the disclosure includes hepatic ultrasound, computed tomographic (CT) abdominal scan, liver magnetic resonance imaging (MRI), body CT scan, and body MRI.

### IV. Kits

Certain aspects of the present disclosure also concern kits containing compositions of the disclosure or compositions to implement methods disclosed herein. In some aspects, kits can be used to evaluate one or more biomarkers. In certain aspects, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some aspects, there are kits for evaluating biomarker activity in a cell.

Kits may comprise components, which may be individually packaged or placed in a container, such as a tube, bottle, vial, syringe, or other suitable container means.

Individual components may also be provided in a kit in concentrated amounts; in some aspects, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. Specifically contemplated are any such molecules corresponding to any biomarker identified herein, which includes nucleic acid primers/primer sets and probes that are identical to or complementary to all or part of a biomarker, which may include noncoding sequences of the biomarker, as well as coding sequences of the biomarker.

In certain aspects, negative and/or positive control nucleic acids, probes, and inhibitors are included in some kit aspects. In addition, a kit may include a sample that is a negative or positive control for methylation of one or more biomarkers.

It is contemplated that any method or composition described herein can be implemented with respect to any other method or composition described herein and that different aspects may be combined. The claims originally filed are contemplated to cover claims that are multiply dependent on any filed claim or combination of filed claims.

Any aspect of the disclosure involving specific biomarker by name is contemplated also to cover aspects involving biomarkers whose sequences are at least 80, 81, 82, 83, 84, 85, 86, 87, 88, 89, 90, 91, 92, 93, 94, 95, 96, 97, 98, 99% identical to the mature sequence of the specified nucleic acid.

Aspects of the disclosure include kits for analysis of a pathological sample by assessing biomarker profile for a sample comprising, in suitable container means, two or more biomarker probes, wherein the biomarker probes detect one or more of the biomarkers identified herein. The kit can further comprise reagents for labeling nucleic acids in the sample. The kit may also include labeling reagents, including at least one of amine-modified nucleotide. poly (A) polymerase, and poly (A) polymerase buffer. Labeling reagents can include an amine-reactive dye.

### A. Detection Kits and Systems

One can recognize that based on the methods described herein, detection reagents, kits, and/or systems can be utilized to detect the biomarkers, including TSG-6, for diagnosing or prognosing an individual. The reagents can be combined into at least one of the established formats for kits and/or systems as known in the art. The kits could also contain other reagents, chemicals, buffers, enzymes, packages, containers, electronic hardware components, etc. The kits/systems could also contain packaged sets of PCR primers, oligonucleotides, arrays, beads, antibodies, or other detection reagents. Any number of probes could be implemented for a detection array. In some aspects, the detection reagents and/or the kits/systems are paired with chemiluminescent or fluorescent detection reagents. Particular aspects of kits/systems include the use of electronic hardware components, such as DNA chips or arrays, or microfluidic systems, for example. In specific aspects, the kit also comprises one or more therapeutic or prophylactic interventions in the event the individual is determined to be in need of.

### EXAMPLES

The following examples are included to demonstrate preferred aspects of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the invention.

### Example 1

### TSG-6 in Cancer-Associated Fibroblasts

The inventors analyzed the tumor and stromal compartment between murine tumor models sensitive to ICT (melanoma) and resistant to ICT (pancreatic tumors) using single cell RNAseq (scRNAseq) and identified a higher expression of TSG-6 in the pancreatic tumors specifically expressed by the cancer-associated fibroblasts (CAFs). Analysis of TCGA data also indicated higher TSG6 (gene name TNFAIP6) in pancreatic tumors as compared to melanoma. TSG-6 expressed by the CAFs interacted with the CD44 expressing suppressive myeloid cells present specifically in the pancreatic tumors. The inventors validated these observations in human pancreatic ductal adenocarcinoma (PDAC) patient samples upon multi immunofluorescence imaging. Further, blocking of TSG-6 in combination with anti-CTLA-4 and anti-PD-1 in vivo showed improved survival by decreasing CD206+VISTA+PDL1+ intratumoral myeloid cells and increasing total and effector CD8T+ cells in the pancreatic tumor models.

### Example 2

### TSG-6 is Expressed by Cancer-Associated Fibroblasts in the ICT-Resistant Pancreatic Tumors

To perform comparative analysis for this study, the inventors utilized B16F10 melanoma and mT4-2D Pancreatic Ductal Adenocarcinoma (PDAC) as the ICT-sensitive and -resistant tumor models, respectively. Mice bearing B16F10 tumors show some survival benefit when treated with anti-CTLA-4 or anti-PD-1 monotherapies and a significant survival benefit when treated with combination therapy. In contrast, mice bearing mT4 tumors show no survival benefit with either monotherapy or combination ICT therapy as compared to the control untreated mice, making these ideal tumor models for the study.

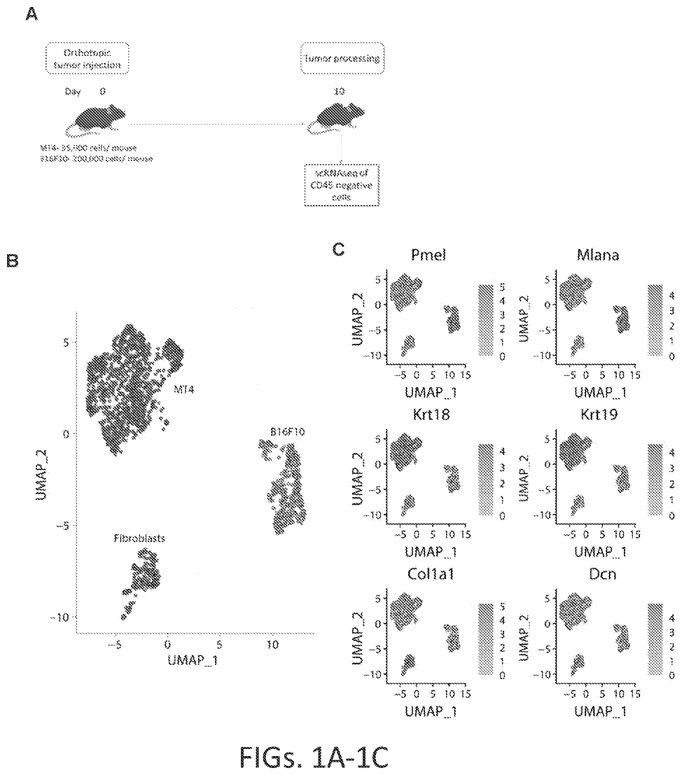

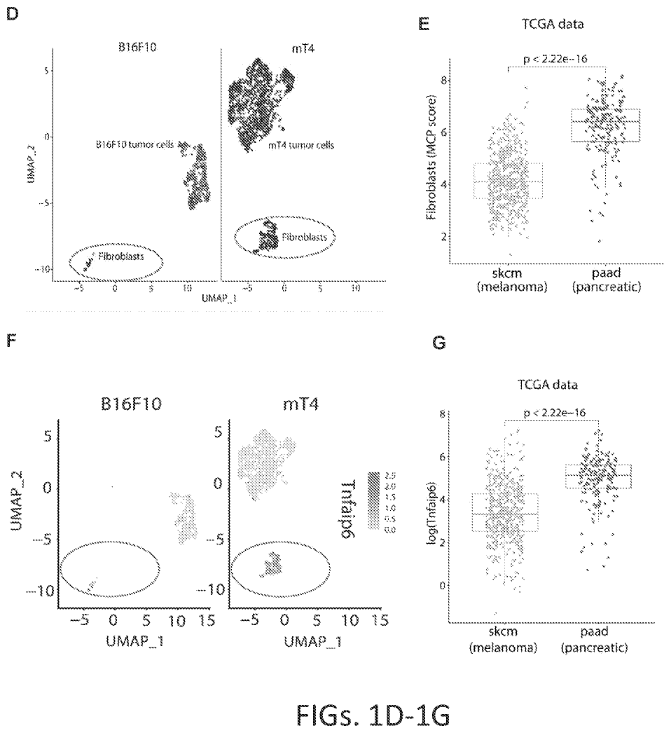

To determine the role of the tumor and stromal compartment in regulating ICT resistance, the inventors performed single cell RNA sequencing (scRNAseq) on CD45 negative cells sorted from orthotopic B16F10 and mT4 tumors (FIG. 1A). Cell clusters obtained were characterized as B16F10 tumor cells, mT4 tumor cells and cancer-associated fibroblasts (CAFs) respectively, based on the gene expression profile (FIGS. 1B, 1C). Of note, the inventors observed higher abundance of CAFs in mT4 tumors than B16F10 tumors (FIG. 1D). Consistent with the murine observations, fibroblast scoring in melanoma and pancreatic patient samples from TCGA datasets also indicated significantly higher fibroblast abundance in pancreatic tumors, thus indicating a possible role of CAFs in ICT resistance (FIG. 1E). Analysis of the gene expression profile in the CAFs highlighted elevated expression of the gene Tnfaip6 (encoding the protein TSG-6) specifically in the mT4 tumors (FIGS. 1F, 1G). To determine significance of this observation in the human settings, the inventors analyzed expression of TNFAIP6/TSG-6 in between melanoma and pancreatic TCGA datasets and observed significantly higher expression in pancreatic tumors, validating the murine observations. Thus, the above data indicates that TSG-6 encoded by gene tnfaip6 which is expressed by the CAFs in the TME in could play a potential role in ICT resistance.

### Example 3

### ICT-Resistant Pancreatic Tumors are Dominated by Suppressive Myeloid Cells Whereas ICT Sensitive Melanoma Tumors are Dominant by Immune Stimulatory Myeloid Cells at Baseline

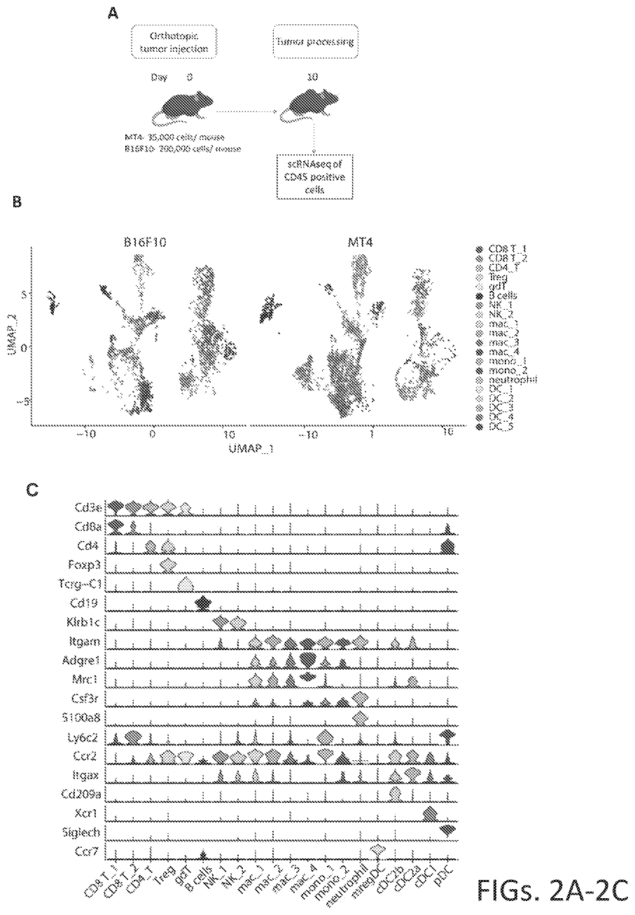

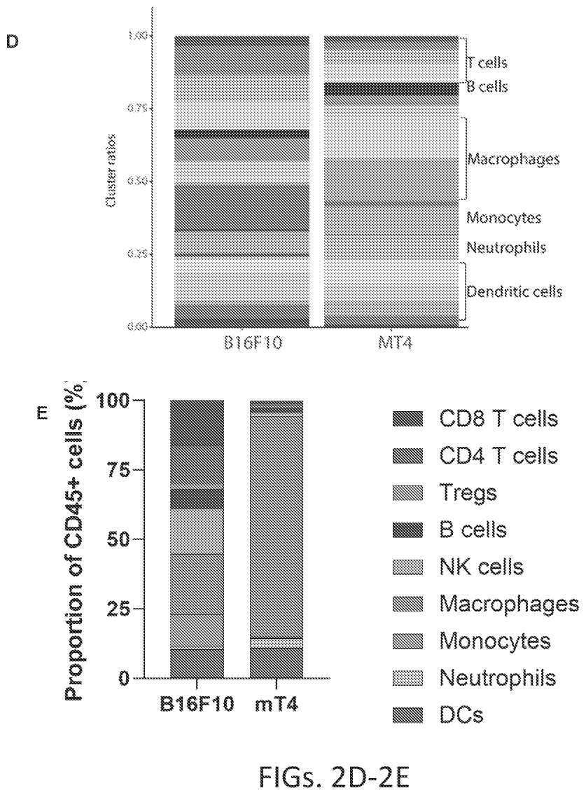

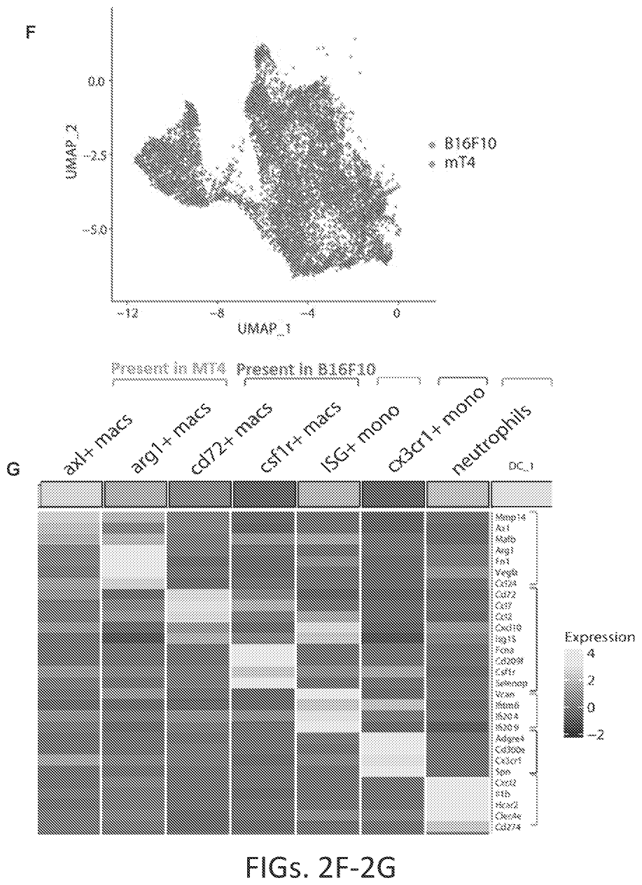

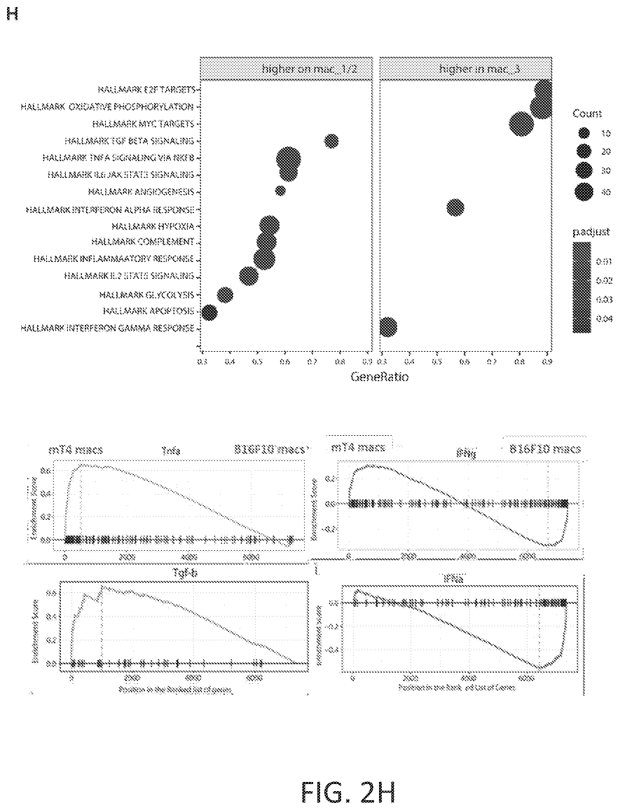

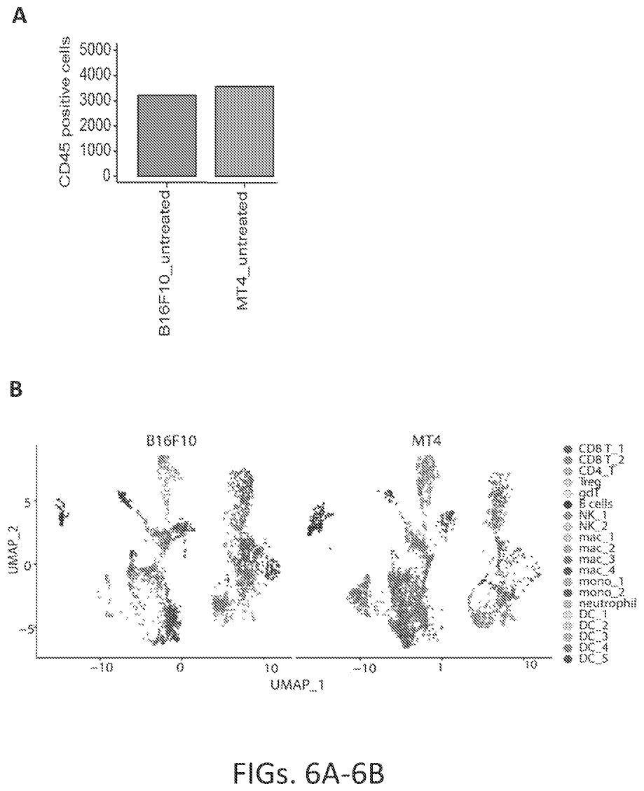

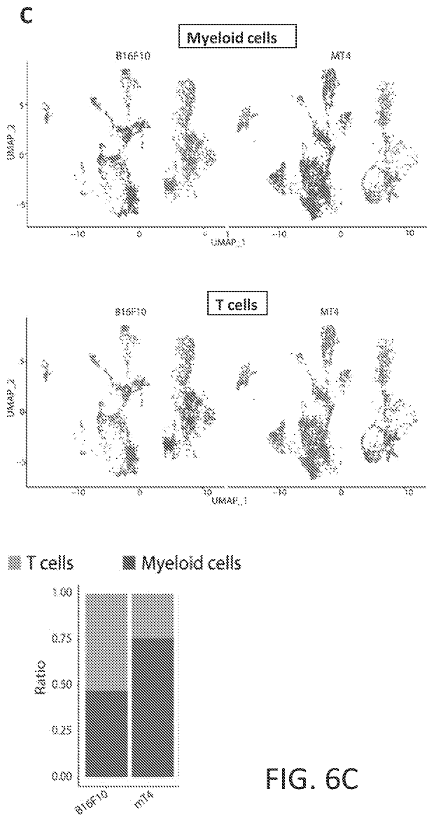

To investigate whether TSG-6 expressed in the TME regulates immune cell polarization and function in tumors, the inventors assessed the differences in the intratumoral immune cell landscape in B16F10 and mT4 tumors (FIG. 2A). A total of 8000 CD45+ cells (FIGS. 6A, 6B) were obtained for analysis which consisted of all major immune subsets of T cells, NK cells, B cells and myeloid cells; including macrophages, dendritic cells (DCs) and neutrophils (FIGS. 2B, 2C, 6C).

While the total DC abundance was similar between the two tumor types, the macrophage/monocyte and neutrophil subsets were more abundant in the mT4 pancreatic tumors whereas the T cells were more abundant in the B16F10 melanoma tumors (FIGS. 2D, 6C). These differences in cell populations were further validated at the protein level using CyTOF (FIG. 2E). Interestingly, these subsets were phenotypically distinct in both tumor models (FIG. 2F) indicating baseline heterogeneity, so the inventors performed in-depth characterization of the cell subsets. Overall, the inventors identified 4 macrophage clusters, 2 monocyte clusters and 1 neutrophil cluster through the analysis (FIGS. 2C, 2G). Two macrophage clusters were abundant primarily in mT4 tumors expressing genes primarily associated with immune suppression (PMID: 15313928). The first expressed mmp14, axl and mafb (denoted as ax1⁺ macs); and the second that expresses vegfa, arg1, ccl24 and fn1 (denoted as arg1⁺ macs) (FIG. 2G). In contrast, the most abundant macrophage cluster in B16F10 tumors, expresses antigen presenting gene (cd72), immune cell migratory chemokines (ccl2, ccl7) and interferon-induced genes (cxcl10, isg15) that the inventors denote as cd72⁺macs. Only a small macrophage cluster present in B16F10 baseline tumors expressed tumor promoting genes (csf1r, selenop) (denoted as csf1r⁺ macs) suggesting that majority of macrophages at baseline B16F10 tumors have pro-inflammatory phenotype. GSEA analysis indicated upregulation of TGF beta signaling, angiogenesis, hypoxia, glycolysis in mT4 macrophages whereas the B16F10 macrophages had upregulated oxidative phosphorylation, interferon gamma and alpha response, highlighting tumor promoting versus tumor inhibiting roles (FIG. 2H). Among the two monocyte subsets observed, one subset was present in both tumor types and expressed interferon-stimulated genes (isg15, ifitm6, ifi204 and ifi209) (ISG⁺ mono) whereas a small subset in B16F10 tumors present depicted similarities with non-classical patrolling monocytes (cx3cr1, spn) (cx3cr1⁺ mono) (PMID: 28348370) (FIG. 2G). The neutrophil cluster were abundantly present in mT4 tumors but not in B16F10 tumors. The subset expressed high levels cd24a, cxcl2, il1b, hcar2, clec4e and cd274 showing similarities with the granulocytic myeloid derived suppressor cells (gMDSC), which have been observed in pancreatic tumor and associate with poor tumor prognosis (FIG. 2G). Together, these observations indicated that the myeloid compartment in the mT4 tumors are significantly immune suppressive and provide a poor environment for generation of an effective immune response in the tumors whereas B16F10 tumors have immune stimulatory myeloid cells indicative of a microenvironment that supports an effective immune response.

### Example 4

### TSG-6 Expressed by CAFs Interact With cd44 Expressing Suppressive Myeloid Cells in ICT Non-Responsive Pancreatic Tumors

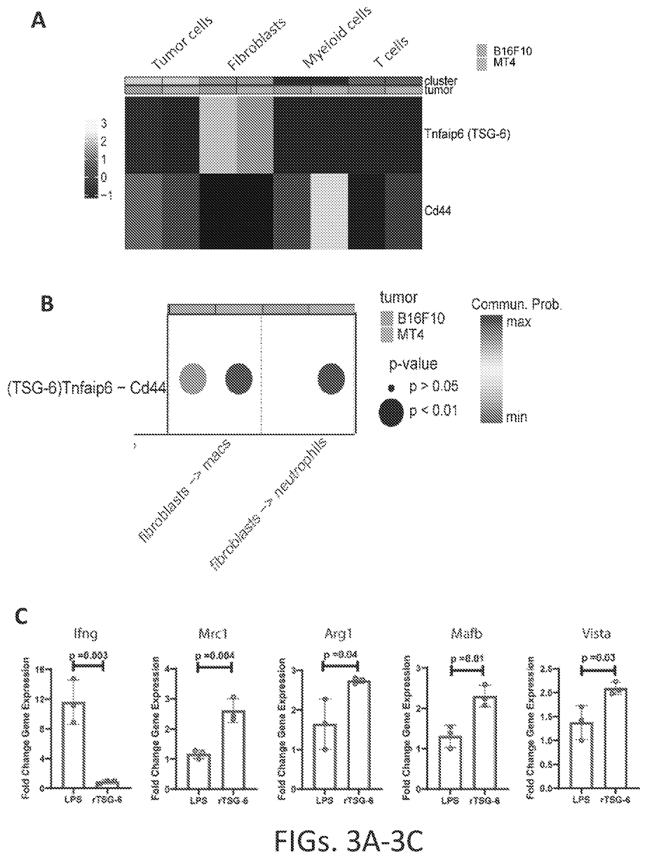

To determine if fibroblast secreted TSG-6 regulates the presence the distinct myeloid subsets between melanoma and pancreatic TME, the inventors performed receptor-ligand studies between the myeloid subsets and fibroblasts. First, the inventors analyzed the relative expression of TSG-6 and its binding partner CD44 in the fibroblasts and macrophage/neutrophil subsets. While TSG-6 was expressed slightly higher in the mT4 tumors as compared to B16F10 tumors, its ligand CD44 was primarily expressed in the macrophage/neutrophil subsets only in the mT4 tumors (FIG. 3A). To determine if this observation correlates with higher interactions between TSG-6 and suppressive myeloid subsets in the mT4 tumors, the inventors performed receptor-ligand interaction studies using CellChat analysis (PMID: 33597522). The analysis indicated strong communication probabilities of fibroblast expressed TSG-6 and myeloid CD44 interaction only in the mT4 tumors and not B16F10 tumors (FIG. 3B). To further evaluate whether TSG-6 regulates the immunosuppressive macrophage phenotype, the inventors performed an in vitro macrophage polarization assay. The inventors stimulated bone marrow-derived macrophages (BMDM) with either LPS or recombinant TSG-6 (rTSG-6) and observed that rTSG-6 increased the expression of Arg1, Mafb, Mrc1, and Vista in the macrophages and suppressed Ifng expression (FIG. 3C). Thus, the above data indicates TSG-6 interacts with suppressive intratumoral myeloid subsets and might have a role to play in resistance to ICT.

### Example 5

### Blocking TSG-6 Reinvigorates the Pancreatic Tumor Microenvironment and Improves ICT Efficacy in Mice

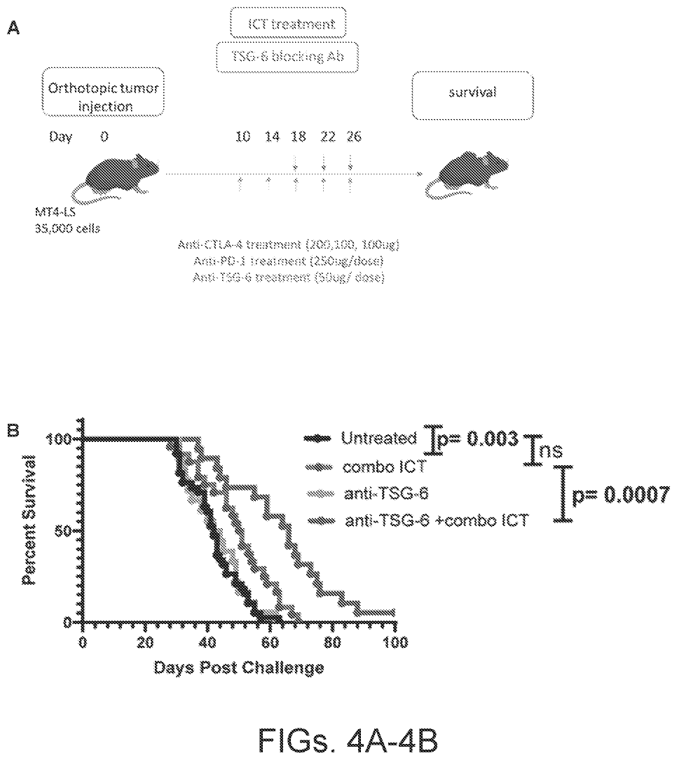

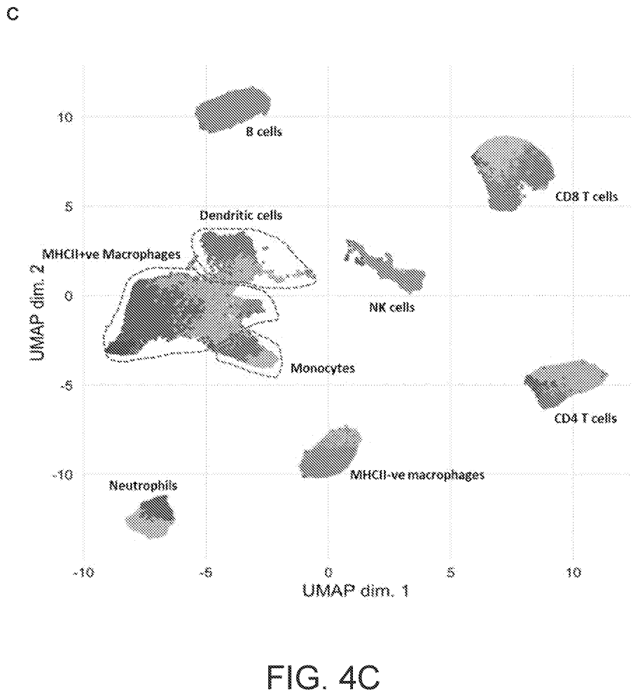

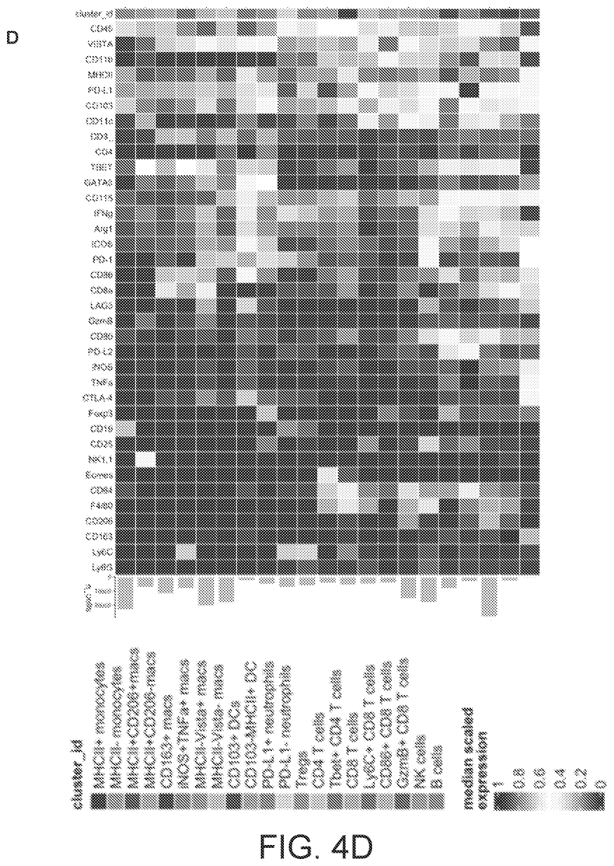

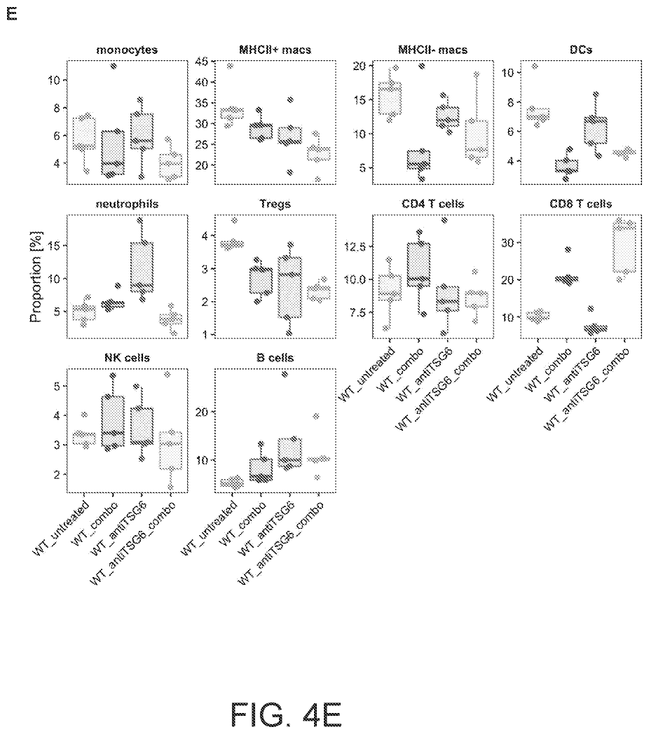

As TSG-6 regulates the phenotype of intratumoral myeloid cells, therapeutic targeting of TSG-6 could repolarize the myeloid cells to pro-inflammatory phenotypes and provide a conducive TME for ICT therapy response. To determine the translational relevance of TSG-6 targeting, the inventors used a commercially available anti-TSG-6 antibody and performed in vivo survival experiments in an orthotopically injected pancreatic tumor model (FIG. 4A). As mT4 are extremely aggressive tumors model, the inventors used the previously established single clone mT4-LS tumor cells derived from the parental line which provide a wider therapeutic window for the experiments (PMID: 34341132). Mice treated with combination of anti-CTLA-4 and anti-PD-1 (which provide no survival benefit in these tumor) and anti-TSG-6 showed significantly longer survival as compared to ICT or monotherapy of anti-TSG-6 (FIG. 4B). To determine the changes induced by the antibody targeting the inventors performed mass cytometry (CyTOF) on the intratumoral immune cells. Interestingly, the inventors observed a decrease in VISTA+ PDL1+ suppressive macrophages and total Ly6G+ neutrophils and a dramatic increase in the CD8T cell abundance in the tumors treated with anti-TSG-6 and ICT antibodies (FIGS. 4C-4E).Thus, above data highlights that neutralizing TSG-6 reprograms the intratumoral myeloid cells and increases CD8 T cell infiltration leading to improved ICT efficacies in ICT non-responsive tumors.

### Example 6

### Analysis of Patient Tumors Validate Preclinical Findings Highlighting Relevance of TSG-6 Targeting to Improve Clinical Responses of ICT

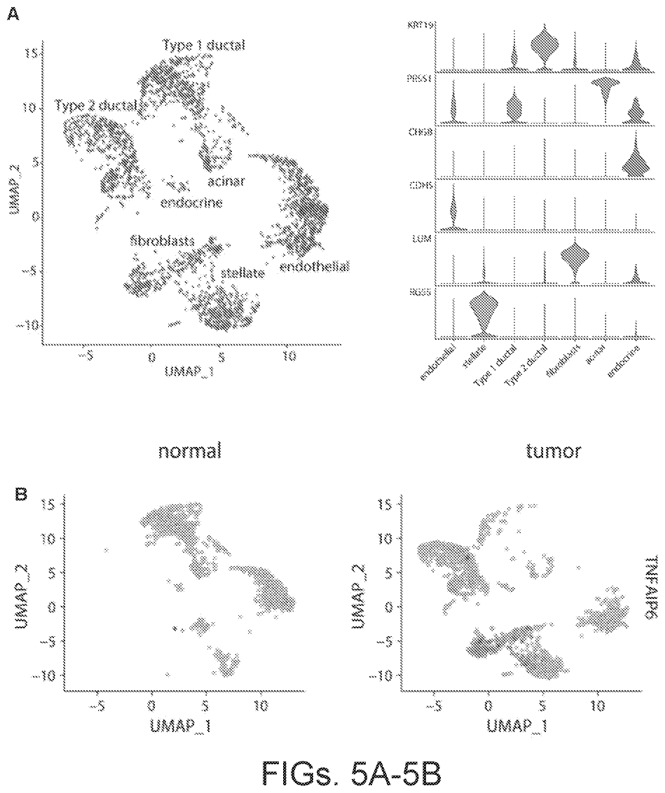

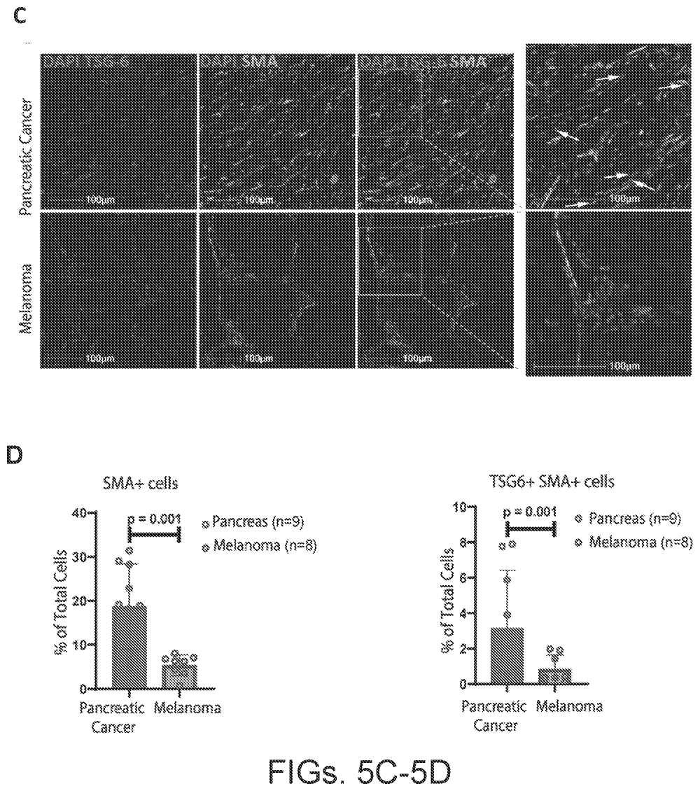

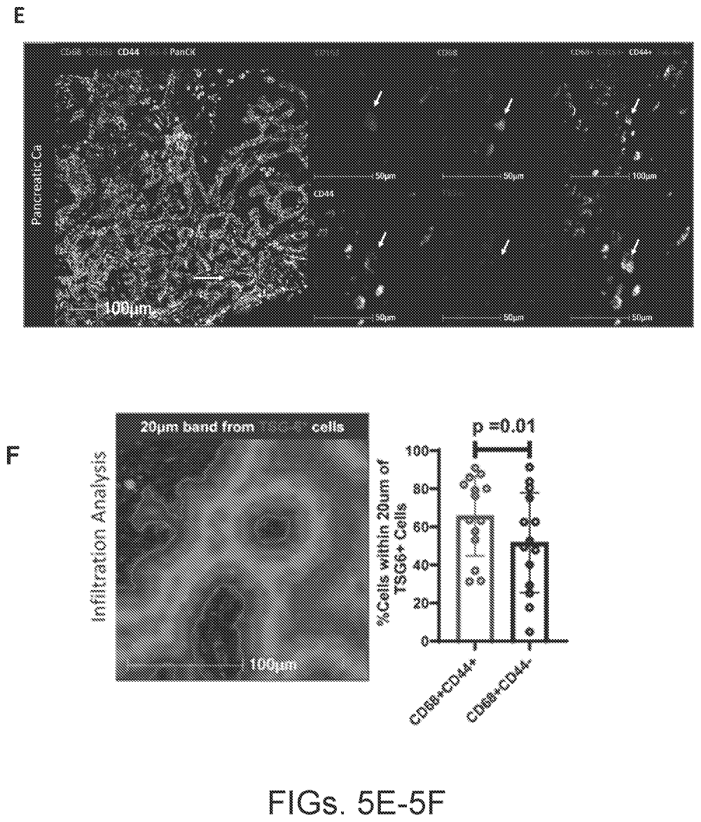

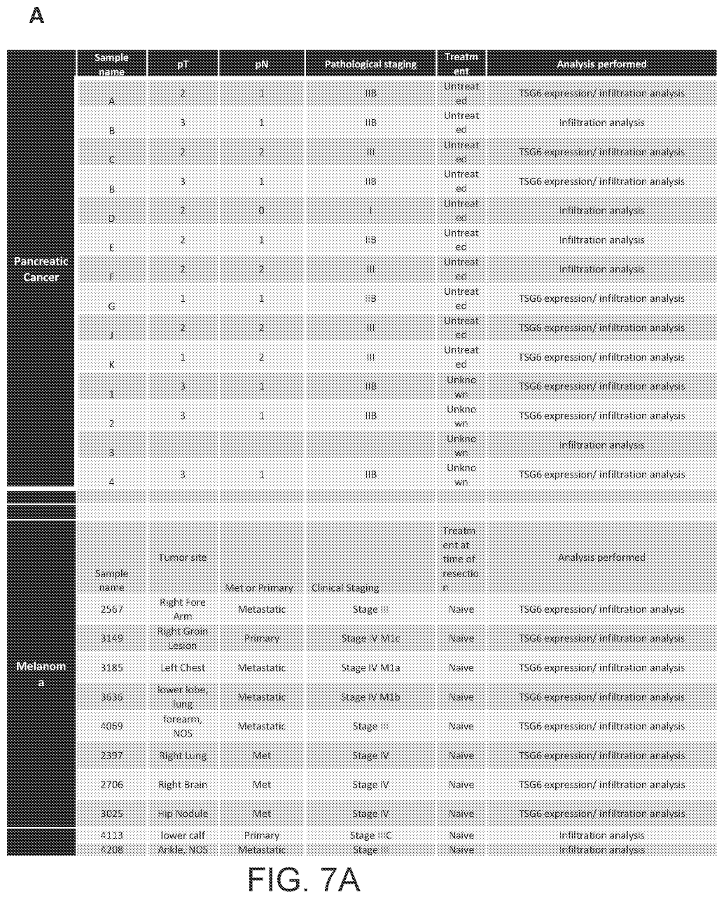

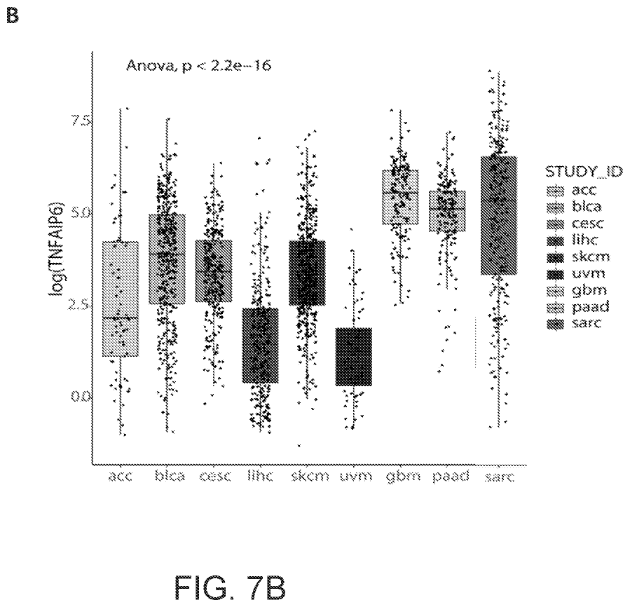

To determine the clinical relevance of TSG-6 targeting in patient tumors, the inventors analyzed a publicly available scRNAseq dataset of baseline PDAC patients and observed elevated expression of TNFAIP6/TSG-6 specifically in CAFs and not normal fibroblasts, consistent with the murine findings (FIGS. 5A, 5B, 7A). Further, to determine if TSG-6 protein levels in the TME correlate with the gene expression data, the inventors performed multi-immunofluorescence (multi-IF) on tissue samples from pancreatic cancer and melanoma patients. They observed higher abundance fibroblasts (staining with SMA) in the pancreatic TME when compared to melanoma tissues. Importantly, these also expressed TSG-6 (FIGS. 5C, 5D).

Next, to assess if the intratumoral TSG-6 interacts with the CD44 expressing myeloid cells in the tumors, the inventors performed multi-immunofluorescence on baseline PDAC patient samples (FIGS. 5E, 5F, 7A). The inventors observed co-localization of CD68+ myeloid cells with TSG-6 in the tumors. Since TSG-6 is a secretory molecule, the inventors also performed spatial proximity analysis and observed more TSG-6 expressing stromal cells closer to CD44+ CD68+ myeloid cells (FIG. 5F), thereby validating the murine findings. Lastly, to determine if TSG-6 could play a role in other ICT non-responsive tumor types, the inventors screened TCGA datasets across difference tumor types and identified similar expression levels of TNFAIP6 in other ICT non-responsive cancers types such as Glioblastoma (GBM) and sarcoma as that in PDAC whereas ICT responsive tumor types such as bladder cancer and liver hepatocellular carcinoma had expression levels similar to melanoma (FIG. 7B). This highlights that TSG-6 could play an important role in other myeloid rich tumor types apart from pancreatic tissues, which should be explored in the future. Overall, the inventors show that TSG-6 is a novel myeloid checkpoint that should be explored in the clinic that could significantly improve response to ICT-non-responsive tumors such as pancreatic cancer and others.

### Example 7

### Materials and Methods for Certain Aspects

Mice: C57BL/6 (5-7 weeks) mice were purchased from the National Cancer Institute (Frederick, MD). All mice were kept in specific pathogen-free conditions in the Animal Resource Center at The University of Texas MD Anderson Cancer Center. Animal protocols were approved by the Institutional Animal Care and Use Committee (IACUC) of The University of Texas MD Anderson Cancer Center.

Cell lines and tumor model: mT4 pancreatic cell line was generous gift from Dr. David A. Tuveson (Cold Spring Harbor Laboratory, NY). mT4 is an organoid cell line generated from mouse pancreata containing PDAC from the KrasLSL-G12D; TrpLSL-R162H; Pdx1-cre mouse model under C57B1/6 background. mT4-LS cell line was generated and generously gifted by Dr. Michael Curran (The University of Texas MD Anderson Cancer Center, Houston, TX). B16F10 melanoma cell line was obtained from Dr. I. Fidler (The University of Texas MD Anderson Cancer Center, Houston, TX). The cells were collected in the logarithmic phase, washed twice with PBS and resuspended in 30% matrigel (Corning)/PBS just before tumor injections. 35,000 cells of mT4 and mT4-LS/50 μl and 200,000 cells of B16F10/100 μl per mouse were injected orthotopically and intradermally, respectively (5 or 10 mice per group).

For pancreatic orthotopic injections, the mice were anesthetized using isoflurane and injected with buprenorphine as prophylactic analgesia (3 mg/ml; i.p.). mT4/mT4-LS cells were surgically implanted in tail of the pancreas using insulin syringes (29 gauge ½). Successful implantation was verified by a clear bubble formation without any intraperitoneal leakage. The peritoneal wall was then closed using dissolvable sutures (brand) and the skin using autoclips (brand). The mice were then kept under a heat lamp till they regained consciousness. Autoclips were removed 10 days post-surgery and mice were euthanized at the indicated time points. For survival experiments, mice were monitored daily post-surgery and euthanized humanely when they became hyperpnea, lethargic, thin or had a hunched posture to determine time of death.

Antibodies and Treatment: Antibodies Anti-CTLA-4 (clone 9H10) and anti-PD-1 (RMP1-14) antibodies were purchased from BioXcell (West Lebanon,NH). Mice were injected intraperitoneally with anti-CTLA-4, anti-PD-1 and combination of anti-PD-1 plus anti-CTLA-4 on day 18 (200 μg/mouse), day 22 (100 μg/mouse) and day 26 (100 μg/mouse) post tumor inoculation. Anti-TSG-6 antibody (catalog no. MAB2104) and isotype control IgG2b antibody (catalog no. MAB004) were purchased from R&D systems (Minneapolis, MN). 50 μg/mouse of each antibody was injected every four days from day 10 prior to tumor injection for a total of five doses.

Mass cytometry (CyTOF): Freshly collected murine tumors from the mice were dissociated with Liberase/DNAse (20 mg/ml) solution, incubated for 30 minutes at 37° C. following which single cell suspensions were made. The cells were given ficoll treatment (histopaque-1193), washed with media and 3 million cells per sample was taken for CyTOF staining. Antibodies were either purchased pre-conjugated from Fluidigm or purchased as purified unconjugated monoclonal antibodies and conjugated in house using MaxPar X8 Polymer kits (Fluidigm) according to the manufacturer's instructions (Supplementary Table 1). Briefly, samples were first stained for viability with 5μM cisplatin in 5% FACS buffer for 3 min at RT, washed thrice with FACS buffer and barcoded using the manufacturer's protocol (Fluidigm). Around 0.75-1 million cells from each sample were then pooled into one tube and stained with cell-surface antibodies for 30 minutes at 4° C. Samples were then washed, fixed (1 hour), permeabilized (eBioscience) and stained with intracellular antibodies for 30 mins at 4° C. Post staining, the samples were washed and incubated with 125 μM Ir intercalator (Fluidigm) in 1.6% PFA/PBS at 4° C. overnight. The cells were then washed with PBS the next day and stored until acquisition. Right before acquisition, samples were washed twice with Milli-Q water, resuspended in water containing EQ 4 element beads (Fluidigm) and run on a Helios mass cytometer (Fluidigm).

### Mass Cytometry Analysis

Data was first demultiplexed using the Fluidigm Debarcoder software. Individual mass cytometry data files (.fcs) were filtered using FlowJo to remove normalization beads, debris, doublets, and dead cells. Remaining analysis was performed in R (version 4.1.0, The R Foundation for Statistical Computing) using R packages ‘cytofkit’ (Chen et al., 2016), ‘flowcore’ (Hahne et al., 2009), and ‘CATALYST’. Processed data was clustered according to the FlowSOM algorithm (k=30) using all cell surface markers and then manually annotated (Van Gassen et al., 2015). Dimensionality reduction was performed using uniform manifold approximation and projection (UMAP) method (Becht et al., 2018). Differential cluster abundances and differential protein expression analyses were performed using novel linear models implemented through ‘edgeR’ (Nikolayeva and Robinson 2014) and ‘limma’ (Diboun et al., 2006), respectively.

Single Cell RNA sequencing: Single cell suspension of melanoma and pancreatic tumors were made using the protocol described above. Single cells were incubated with a surface staining cocktail of fluorescently conjugated antibodies, which included CD45 Pacific Blue (clone 30-F11, Biolegend, 103126), and live/dead discrimination viability dye Pacific Orange (Invitrogen, L34968). CD45+and CD45-cells were sorted into RPMI with 5% FBS using a FACS AriaFusion cell sorter (BD). Cell suspensions were assessed for cell concentration and viability using Life Technologies Countess 3 FL cell counter using 0.4% trypan blue exclusion staining (dead cells more permeable to staining blue). Samples passing QC fall in the concentration range for their cell target capture and have a viability of at least 70% or higher. Reagents, consumables, reaction master mixes, reaction volumes, cycling numbers, cycling conditions, and clean up steps were completed following 10X Genomics' Next GEM 3′ SCRNAseq V3.1 protocol (CG000204_ChromiumNextGEMSingleCell3_v3.1_Rev_D). QC steps after cDNA amplification and library preparation steps were carried out by running ThermoFisher Qubit HS dsDNA Assay along with Agilent HS DNA Bioanalyzer for concentration and quality assessments, respectively. Equal amounts of each uniquely-indexed sample library were pooled together. The resultant pool was verified for concentration via qPCR using a KAPA Biosystems KAPA Library Quantification Kit. The pool was sequenced using a NovaSeq6000 sequencer depending on the total number of samples, the target cell numbers/sample, and read depth of 50,000 read pairs/cell. The run parameters used were 28 cycles for read 1, 91 cycles for read2, 8 cycles for index 1, and 0 cycles for index2 as stipulated in the protocol mentioned above. Raw sequencing data (fastq file) was demultiplexed and analyzed using 10X Genomics Cell Ranger software utilizing standard default settings and the cellranger count command to generate html QC metrics and cloupe files for each sample. Further analysis can be achieved using the cLoupe files in 10X Genomics Loupe Browser software.

### Pre-Processing of scRNA-Seq Data

An initial seurat objects was created by merging the ‘filtered_feature_bc_matrix’ from each sample in an experiment. The inventors kept all features present in at least 3 cells. Cells were required to have greater than 200 and less than 6,000 unique features as well as a mitochondrial gene fraction less than 0.25. The RNA data was then normalized, and cell-cycle genes were regressed out by regularized negative binomial regression (SCTransform). Data was the subjected to linear dimensionality reduction using PCA. The Uniform Manifold Approximation and Projection (UMAP) dimensional reduction technique was then carried out using the first 30 principal components. The 20 nearest neighbors were estimated using the first 30 principal components and clustered. Clusters were manually annotated by evaluating the differentially expressed genes for each individual cluster against all other cluster

Cluster pathway analysis and GSEA: Wilcox tests were performed to calculate differential gene expression between the clusters or conditions of interest. Gene ranks were calculated using the resulting log2 fold change. The inventors evaluated the HALLMARK subset of Canonical Pathways in MSigDB v 7.1 (Subramanian et al., 2005) and the inventors considered pathways with Benjamini-Hochberg adjusted p-values <0.05 to be significant.

Cell-cell interaction analysis: To identify potential cell-cell interactions between the myeloid cell subsets and fibroblasts the inventors used CellChat (Jin et al. 2021); a tool that employs pattern recognition and network analysis to infer the existence of intercellular communication from single-cell RNA-sequencing.

TSG-6 stimulated bone marrow derived macrophages (BMDM) and RT-qPCR Gene Expression: BMDM were isolated from the femurs of wild type C57/BL6 mice in ice cold complete RPMI 1640 media. Both epiphyses were removed before the bones were placed in sterile microfuge tubes and centrifuged at 500×g for 5 minutes at 4° C. to extrude the bone marrow. The collected bone marrow was homogenized by pipetting followed by RBC lysis. The single cell suspension of bone marrow cells obtained, was counted in an automated Vicell cell counter before 2.0e6 cells/well were cultured in Iscove's Modified Dulbecco's Medium (IMDM) supplemented with 10% FBS and 10 ng/ml Macrophage colony-stimulating factor (M-CSF) (Biolegend, Cat #576404)) in a 12 well plate. On Day 3, cells were resuspended in fresh growth media and on Day 7, cells were subjected to fresh complete IMDM containing 100 ng/ml LPS (InvivoGen, Cat #tlrl-eblps) and 0.5 mg/mL recombinant TSG6 (R&D Systems, Cat #2326-TS-050) and left in culture for 2 days. Supernatants were removed and adhered macrophages were subjected to Accutase cell detachment solution following manufacturers protocol (Innovative Cell Technologies, 07920). Macrophages were resuspended and centrifuged at 350×g for 8 minutes. Cell pellets were lysed with 350 uL buffer RLT lysis buffer (Qiagen, 74104) supplemented with B-ME and subjected to RNA extraction using RNA mini extraction kit (Qiagen, 74104). cDNA was synthesized using the NEB LunaScript RT SuperMix Kit (E3010L) and 30 ng of total cDNA was used for each 20 uL qPCR reaction. Taqman gene expression Real-Time PCR assays were run using manufacturers protocol and gene targets were compared to GAPDH housekeeping expression (GAPDH, Mm05724508_g1; IFNg, Mm01168134_m1; Mrc1, Mm01329359_m1; Arg1, Mm00475988_m1; Mafb, Mm00627481_s1, VISTA, Mm00472312_m1).

Multiplex immunofluorescence assay and analysis: Using the Opal multiplex immunofluorescence staining protocol 54 on a RX-BOND (Leica) autostainer, pancreatic cancer (n=4) tissue sections were stained for CD68 (companyDako-Agilent, PGM-1, 1:25 Dilution), CD163 (companyLeica Biosystems, 10D6, 1:50 Dilution), CD44 (companyCell Signaling Technologies, E7K27, 1:800 Dilution), TSG-6 (companyNovusbio, 38637, 1:40 Dilution), and PanCK (Dako-Agilent company, AE1/3, 1:500 Dilution). Subsequent visualization was performed using Akoya Opal fluorophores (480, 690, 620, 520, 570 respectively), DAPI (1:2000 dilution) and cover-slipped using Vectashield Hardset mounting medium. Slides were scanned using a Vectra/Polaris slide scanner (PerkinElmer) and images acquired at ×20 magnification were spectrally unmixed using Inform software (Akoya). Cases were analyzed for cell density and infiltration analysis using HALO software (Indica labs, HighPlex FL v4.04) to determine the cells/mm2 area, distance of CD68+CD44+ or CD68+CD44− cells from TSG-6+ cells. The data were plotted using Prism v.8.0 (GraphPad Software). Statistical significance was calculated using a Wilcoxon rank-sumTwo Tailed/Paired t-test. P<0.05 was considered statistically significant.

Statistical analyses: For all murine experiments, data are representative of at least two to three independent experiments with 5-10 mice in each in vivo experiment. The data are expressed as mean±standard error of the mean (SEM) and were analyzed using Prism 7.0 statistical analysis software (GraphPad Software, La Jolla, CA). Student t-tests (two tailed) and ANOVA were used to identify significant differences (p<0.05) between treatment groups.

Data availability: Raw reads for the single cell RNA sequencing will be deposited in European Genome-phenome Archive.

All of the methods disclosed and claimed herein can be made and executed without undue experimentation in light of the present disclosure. While the compositions and methods of this invention have been described in terms of preferred aspects, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Mittal et al. “TNFα-stimulated gene-6 (TSG6) activates macrophage phenotype transition to prevent inflammatory lung injury” PNAS, 113 (50): E8151-E8158 (2016).
Liu et al. “TSG-6 promotes Cancer Cell aggressiveness in a CD44-Dependent Manner and Reprograms Normal Fibroblasts to create a Pro-metastatic Microenvironment in Colorectal Cancer” Int J Biol Sci, 18(4): 1677-1694 (2022).
Dyer et al. “TSG-6 Inhibits Neutrophil Migration via Direct Interaction with the Chemokine CXCL8” J Immunol, 192 (5): 2177-2185 (2014).
Choi et al. “Anti-inflammatory protein TSG-6 secreted by activated MSCs attenuates zymosan-induced mouse peritonitis by decreasing TLR2/NF-KB signaling in resident macrophages” Blood, 118 (2): 330-338 (2011).
Spinelli et al. “Hyaluronan preconditioning of monocytes/macrophages affects their angiogenic behavior and regulation of TSG-6 expression in a tumor type-specific manner” FEBS, 286 (17): 3433-3449 (2019).

What is claimed is:

1. A method of detecting TNF stimulated gene 6 (TSG-6) secretion in a population of cancer-associated fibroblasts (CAFs), the method comprising the step of detecting TSG-6 in a biological sample comprising CAF secretions from an individual, wherein the individual has a cancer that is a candidate for an immune checkpoint therapy.

2. The method of claim 1, wherein the biological sample comprises CAFs.

3. The method of claim 1 or 2, wherein the biological sample comprising CAF secretions comprises a tumor biopsy.

4. The method of any one of claims 1-3, wherein the biological sample is enriched for fibroblasts.

5. The method of any one of claims 1-4, wherein the biological sample is enriched for CAFs.

6. The method of claim 1 or 3, wherein the biological sample is prepared for immunohistochemistry.

7. The method of any one of claims 1-6, wherein detecting TSG-6 comprises detecting TSG-6 protein.

8. The method of claim 7, wherein detecting TSG-6 protein comprises detecting by immunohistochemistry.

9. The method of claim 7 or 8, wherein the detecting TSG-6 protein comprises detecting by Western blot, ELISA, cytometry, chromatography, mass spectrometry, and/or immunoprecipitation.

10. The method of any one of claims 1-9, wherein detecting TSG-6 comprises detecting TSG-6 mRNA.

11. The method of claim 10, wherein the detecting TSG-6 mRNA comprises reverse transcription polymerase chain reaction (RT-PCR).

12. The method of claim 11, wherein the RT-PCR is quantitative RT-PCR.

13. The method of any one of claims 10-12, wherein the detecting TSG-6 mRNA comprises RNA sequencing.

14. The method of claim 13, wherein the RNA sequencing comprises single cell RNA sequencing.

15. The method of any one of claims 1-14, wherein the immune checkpoint therapy comprises an anti-PD1 composition and/or an anti-CTLA4 composition.

16. The method of any one of claims 1-15, wherein the detecting TSG-6 comprises detecting an amount of TSG-6 that is greater than a reference level.

17. The method of any one of claims 1-15, wherein the detecting TSG-6 comprises detecting an amount of TSG-6 that is substantially equal to or less than a reference level.

18. The method of claim 16 or 17, wherein the reference level is determined by measuring TSG-6 in two or more biological samples from the individual.

19. The method of claim 16 or 17, wherein the reference level comprises an amount of TSG-6 found in an individual responsive to an immune checkpoint therapy or a healthy individual.

20. A method of treating a cancer in an individual, the method comprising the step of administering a non-immune checkpoint therapy to an individual who has been determined to have cancer-associated fibroblasts (CAFs) that secrete TSG-6 in an amount greater than a reference level.

21. The method of claim 20, wherein the non-immune checkpoint therapy comprises a chemotherapy, a non-immune checkpoint immunotherapy, a cell therapy, a biologic, a steroid, radiation, and/or surgery.

22. The method of claim 20 or 21, wherein the reference level is a detectable amount of TSG-6 secreting CAFs present in the individual prior to administering the non-immune checkpoint therapy.

23. The method of claim 20 or 21, wherein the reference level is determined by measuring TSG-6 secreting CAFs in two or more biological samples from the individual.

24. The method of claim 20 or 21, wherein the reference level comprises an amount of TSG-6 secreting CAFs found in an individual responsive to an immune checkpoint therapy or a healthy individual.

25. A method of treating a cancer in an individual, the method comprising the step of administering an immune checkpoint therapy to an individual who has been determined to have cancer-associated fibroblasts (CAFs) that secrete TSG-6 in an amount substantially equal to or less than a reference level.

26. The method of claim 25, wherein the reference level is a detectable amount of TSG-6 secreting CAFs present in the individual prior to administering the non-immune checkpoint therapy.

27. The method of claim 25, wherein the reference level is determined by measuring TSG-6 secreting CAFs in two or more biological samples from the individual.

28. The method of claim 25, wherein the reference level comprises an amount of TSG-6 secreting CAFs found in an individual responsive to an immune checkpoint therapy or a healthy individual.

29. A method for evaluating effectiveness of an immune checkpoint therapy in a cancer patient, the method comprising the step of detecting TSG-6 in a biological sample to obtain a measurement of TSG-6 and comparing the measurement of TSG-6 to a reference.

30. The method of claim 29, wherein the immune checkpoint therapy comprises an anti-PD1 composition and/or an anti-CTLA4 composition.

31. The method of claim 29 or 30, wherein the biological sample comprises cancer associated fibroblasts (CAFs).

32. The method of any one of claims 29-31, wherein the biological sample comprises a tumor biopsy.

33. The method of any one of claims 29-32, wherein the biological sample is enriched for fibroblasts.

34. The method of any one of claims 29-33, wherein the biological sample is enriched for CAFs.

35. The method of any one of claims 29-32, wherein the biological sample is prepared for immunohistochemistry.

36. The method of any one of claims 29-35, wherein detecting TSG-6 comprises detecting TSG-6 protein.

37. The method of claim 36, wherein detecting TSG-6 protein comprises detecting by immunohistochemistry.

38. The method of claim 36 or 37, wherein the detecting TSG-6 protein comprises detecting by Western blot, ELISA, cytometry, chromatography, mass spectrometry, and/or immunoprecipitation.

39. The method of any one of claims 29-38, wherein detecting TSG-6 comprises detecting TSG-6 mRNA.

40. The method of claim 39, wherein the detecting TSG-6 mRNA comprises reverse transcription polymerase chain reaction (RT-PCR).

41. The method of claim 40, wherein the RT-PCR is quantitative RT-PCR.

42. The method of any one of claims 39-41, wherein the detecting TSG-6 mRNA comprises RNA sequencing.

43. The method of claim 42, wherein the RNA sequencing comprises single cell RNA sequencing.

44. The method of any one of claims 29-43, wherein the reference is a detectable amount of TSG-6 secreting CAFs present in the patient.

45. The method of any one of claims 29-44, wherein the reference is determined from measuring TSG-6 secreting CAFs in two or more biological samples from the individual.

46. The method of any one of claims 29-44, wherein the reference comprises an amount of TSG-6 secreting CAFs found in an individual responsive to an immune checkpoint therapy or a healthy individual.

47. A method of treating a cancer in an individual, the method comprising the step of administering a composition comprising a therapeutically effective amount of an anti-TSG-6 agent.

48. The method of claim 47, wherein the individual has been determined to have cancer-associated fibroblasts (CAFs) that secrete TSG-6 in an amount greater than a reference level.

49. The method of claim 48, wherein the reference level is a detectable amount of TSG-6 secreting CAFs present in the individual.

50. The method of claim 48, wherein the reference level is determined by measuring TSG-6 secreting CAFs in two or more biological samples from the individual.

51. The method of claim 48, wherein the reference level comprises an amount of TSG-6 secreting CAFs found in an individual responsive to an immune checkpoint therapy or a healthy individual.

52. The method of any one of claims 47-51, wherein the anti-TSG-6 composition comprises an antibody targeting TSG-6, an RNA interference composition targeting TSG-6, and/or a small molecule capable of inhibiting TSG-6.

53. The method of any one of claims 47-52, further comprising administering a therapeutically effective amount of an immune checkpoint therapy.

54. The method of claim 53, wherein the composition comprises the immune checkpoint therapy.

55. The method of claim 53, wherein the composition does not comprise the immune checkpoint therapy.

56. The method of claim 53, wherein the composition and immune checkpoint therapy are given concurrently.

57. The method of claim 53, wherein the composition and immune checkpoint therapy are given sequentially.

58. The method of any one of claims 53-56, wherein the immune checkpoint therapy comprises an anti-PD1 composition and/or an anti-CTLA4 composition.

59. The method of any one of claims 47-58, wherein the cancer is refractory to an immune checkpoint therapy.

60. The method of any one of claims 47-59, wherein the cancer is a myeloid-rich cancer.

61. The method of any one of claims 47-60, wherein the cancer is a pancreatic cancer, a glioblastoma, and/or a prostate cancer.

62. The method of any one of claims 47-59, wherein the cancer is a melanoma.

63. A method for determining a treatment for an individual with cancer, the method comprising the step of administering a non-checkpoint inhibitor therapy to an individual found to have an amount of TSG-6 secreting cancer-associated fibroblasts (CAFs) greater than a reference.

64. The method of claim 63, wherein the detectable amount of TSG-6 secreting CAFs is found by measuring the expression of TSG-6 in at least one biological sample from the individual.

65. The method of claim 64, wherein the biological sample comprises a tumor biopsy.

66. The method of claim 64 or 65, wherein the biological sample is enriched for fibroblasts.

67. The method of any one of claims 64-66, wherein the biological sample is enriched CAFs.

68. The method of claim 64 or 65, wherein the biological sample is prepared for immunohistochemistry.

69. The method of any one of claims 63-68, wherein the reference is a detectable amount of TSG-6 secreting CAFs present in the individual.

70. The method of claim 69, wherein the reference is determined from measuring TSG-6 secreting CAFs in two or more biological samples from the individual.

71. The method of claim 69, wherein the reference comprises an amount of TSG-6 secreting CAFs found in an individual responsive to an immune checkpoint therapy or a healthy individual.
